(19) United States
(12) Patent Application Publication
(10) Pub. No.: US 2026/0200901 A1
(43) Pub. Date: Jul. 16, 2026

(54) NOVEL COMPOUNDS AS MODULATORS OF NLRP3 INHIBITION

(71) Applicant: Hoffmann-La Roche Inc., Little Falls, NJ (US)

(72) Inventors: Lea Aurelie BOUCHE, Basel (CH); Wolfgang GUBA, Müllheim (DE); Georg JAESCHKE, Basel (CH); Heather Jennifer JOHNSTON, Nottingham (GB); Stefanie Katharina MESCH, Basel (CH); Jonathan Martin SHANNON, Nottingham (GB); Sandra STEINER, Sursee (CH)

(73) Assignee: Hoffmann-La Roche Inc., Little Falls, NJ (US)

(21) Appl. No.: 19/135,999

(22) PCT Filed: Dec. 5, 2023
(86) PCT No.: PCT/EP2023/084200

§ 371 Date: Jun. 5, 2025

(30) Foreign Application Priority Data
Dec. 7, 2022 (EP) 22211843.2
May 16, 2023 (EP) 23173506.9

(51) Int. Cl. C07D 405/14; A61K 31/53; A61K 31/5377; C07D 413/14

(52) U.S. Cl. CPC C07D 405/14; A61K 31/53; A61K 31/5377; C07D 413/14

(57) ABSTRACT

The invention relates to novel compounds having the general formula (I), wherein R¹, R², R³, R⁴, R⁵, and R⁶, are as described herein, composition including the compounds and methods of using the compounds.

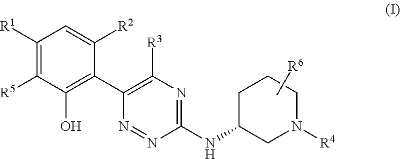

### FIELD OF THE INVENTION

The present invention relates to organic compounds useful for therapy and/or prophylaxis in a mammal, and in particular to compounds that modulate NLRP3 inhibition.

The present invention provides novel compounds of formula I

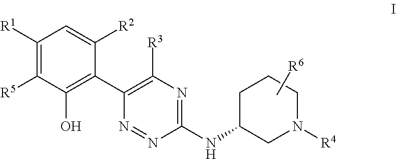

wherein
R¹ and R⁵, and the atoms to which they are bonded, form either
   i. a 4-6 membered heterocycle ring comprising a single O heteroatom, or
   ii. a 4-5 membered cycloalkyl ring;
R² and R³ are selected from H and alkyl, wherein only one of R² or R³ can be H or alkyl;
R⁴ is selected from
   i. alkyl,
   ii. hydroxyalkyl,
   iii. alkoxyalkyl,
   iv. cycloalkyl substituted with hydroxy or alkoxy,
   v. cycloalkylalkyl substituted with hydroxyl, alkoxy, or —COOH,
   vi. a 4-6 membered heterocycle comprising a single O heteroatom,
   vii. (CH₂)ₙC(O)OH, (CH₂)ₙC(O)OCH₃, or (CH₂)ₙC(O)NHCH₃, wherein n is 2 or 3,
   viii. heteroarylalkyl, wherein the heteroaryl in heteroarylalkyl is a tetrazole, oxadiazole or an oxazole heteroaryl,
   ix. heteroarylalkyl, wherein the heteroaryl in heteroarylalkyl is a pyrazole,
   x. —(CH₂)ₙS(O)₂CH₃ or (CH₂)ₙCN wherein n is 3,
   xi. —(CH₂)ₙC(O)NR′R″, wherein n is 3 and R′ and R″ are both CH₃, R′ is CH₃ and R″ is a hydroxylalkyl, or R′ and R″ together with the N to which they are connected form either
      a. a 5 member heterocycle ring, wherein the heterocycle ring is optionally substituted with OH, or
      b. a 6 member heterocycle ring additionally comprising 1 O heteroatom;
R⁶ is H or —OH;
   wherein R⁴ can only be alkyl when R⁶ is —OH;
and pharmaceutically acceptable salts.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signaling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-p-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds. Further, in developing a NLRP3 inhibitor for treating peripheral indications, it is advantageous to minimize the exposure of an NLRP3 inhibiting compound in the brain relative to systemic exposure to minimize the risk of potential side effects in the central nervous system (CNS). The compounds of formula I achieve this by showing an increased efflux in a transcellular assay expressing active P-gp protein and/or reduced permeability. P-gp (P-glycoprotein) is an important transporter that is expressed in the capillary endothelial cells composing the blood-brain barrier and blood-testis barrier, where it pumps xenobiotics back into the capillaries and limiting brain exposure.

### SUMMARY OF THE INVENTION

The present invention provides novel compounds of formula I

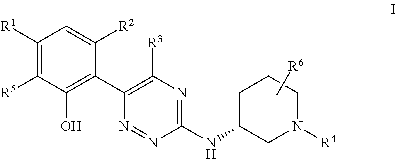

wherein
R¹ and R⁵, and the atoms to which they are bonded, form either
   i. a 4-6 membered heterocycle ring comprising a single O heteroatom, or
   ii. a 4-5 membered cycloalkyl ring;
R² and R³ are selected from H and alkyl, wherein only one of R² or R³ can be H or alkyl;
R⁴ is selected from
   i. alkyl,
   ii. hydroxyalkyl,
   iii. alkoxyalkyl,
   iv. cycloalkyl substituted with hydroxy or alkoxy,
   v. cycloalkylalkyl substituted with hydroxyl, alkoxy, or —COOH,
   vi. a 4-6 membered heterocycle comprising a single O heteroatom,
   vii. (CH₂)ₙC(O)OH, (CH₂)ₙC(O)OCH₃, or (CH₂)ₙC(O)NHCH₃, wherein n is 2 or 3,
   viii. heteroarylalkyl, wherein the heteroaryl in heteroarylalkyl is a tetrazole, oxadiazole or an oxazole heteroaryl,
   ix. heteroarylalkyl, wherein the heteroaryl in heteroarylalkyl is a pyrazole;
   x. —(CH₂)ₙS(O)₂CH₃ or (CH₂)ₙCN wherein n is 3,
   xi. —(CH₂)ₙC(O)NR′R″, wherein n is 3 and R′ and R″ are both CH₃, R′ is CH₃ and R″ is a hydroxylalkyl, or R′ and R″ together with the N to which they are connected form either
      a. a 5 member heterocycle ring, wherein the heterocycle ring is optionally substituted with OH, or
      b. a 6 member heterocycle ring additionally comprising 1 O heteroatom;
R⁶ is H or —OH,
   wherein R⁴ can only be alkyl when R⁶ is —OH;
and pharmaceutically acceptable salts.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl. Other particular alkyl group is propyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy.

The term “alkoxyalkyl” denotes an alkyl group wherein one of the hydrogen atoms of the alkyl group have been replaced by an alkoxy group. Examples of alkoxyalkyl are methoxymethyl and methoxyethyl. Particular examples of alkoxyalkyl are methoxyethyl and methoxypropyl.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. Examples of cycloalkyl groups include cyclopropyl, cyclobutyl, cyclopentyl, cyclohexyl, and the like.

The term “cycloalkylalkyl” denotes an alkyl group wherein at least one of the hydrogen atoms of the alkyl group is replaced by a cycloalkyl group. Examples of cycloalkylalkyl include cyclopropylmethyl and cyclobutylmethyl. Particular cycloalkylalkyl is cyclopropylmethyl.

The term “cyano” denotes a —C—N group.

The term “heterocycle ring” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Examples for monocyclic saturated heterocycle rings are azetidinyl, diazepanyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, morpholinyl, and piperazinyl. Examples of polycyclic saturated heterocycle rings are azaspiroheptanyl, diazaspiroheptanyl, azaspirooctanyl, diazospirooctanyl, diazaspirononanyl, oxaazaspirooctanyl, and oxadiazaspirononanyl. A particular example of a heterocycle ring is piperidinyl. Another particular example of a heterocycle ring is oxetanyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl group include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridinyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, and benzothiophenyl. A particular example of a heteroaryl group is oxazole.

The term “heteroarylalkyl”, denotes an alkyl group wherein one of the hydrogen atoms of the alkyl group has been replaced by a heteroaryl group. Examples are oxyzolylethyl, tetrazolylethyl, and oxadiazolylethyl. Particular example is oxazolylethyl. Other examples are pyrazolylethyl, tetrazolylpropyl, oxazolylpropyl and tetrazolylethyl.

The term “hydroxy” or “hydroxyl” denotes a —OH group.

The term “hydroxyalkyl” denotes an alkyl group wherein at least one of the hydrogen atoms of the alkyl group has been replaced by a hydroxy group. Examples of hydroxyalkyl include hydroxymethyl, hydroxyethyl, hydroxypropyl, 2-hydroxy-1-propyl, 2-hydroxy-2-methyl-1-propyl, 3-hydroxy-1-propyl, and the like. Particular examples of hydroxyalkyl are hydroxyethyl and hydroxypropyl.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ and R⁵, and the atoms to which they are bonded, form a 4-6 membered heterocycle ring comprising a single O heteroatom.

An embodiment of the present invention provides compounds according to formula I as described herein, wherein R¹ and R⁵, and the atoms to which they are bonded, form a 4-5 membered heterocycle ring comprising a single O heteroatom.

An embodiment of the present invention provides compounds according to formula I as described herein, wherein R¹ and R⁵, and the atoms to which they are bonded, form a 5 membered heterocycle ring comprising a single O heteroatom.

An embodiment of the present invention provides compounds according to formula I as described herein, wherein R² is H and R³ is alkyl.

An embodiment of the present invention provides compounds according to formula I as described herein, wherein R² is H and R³ is methyl.

An embodiment of the present invention provides compounds according to formula I as described herein, wherein R⁴ is selected from
i. alkyl,
ii. hydroxyalkyl,
iii. alkoxyalkyl,
iv. cycloalkyl substituted with hydroxy or alkoxy,
v. cycloalkylalkyl substituted with hydroxyl, alkoxy, or —COOH,
vi. a 4-6 membered heterocycle comprising a single O heteroatom,
vii. (CH₂)ₙC(O)OH, (CH₂)ₙC(O)OCH₃, or (CH₂)ₙC(O)NHCH₃, wherein n is 2 or 3,
viii. heteroarylalkyl, wherein the heteroaryl in heteroarylalkyl is an oxazole heteroaryl,
ix. heteroarylalkyl, wherein the heteroaryl in heteroarylalkyl is a pyrazole,
x. —(CH₂)ₙS(O)₂CH₃ or (CH₂)ₙCN wherein n is 3,
xi. —(CH₂)ₙC(O)NR′R″, wherein n is 3 and R′ and R″ are both CH₃, R′ is CH₃ and R″ is a hydroxylalkyl, or R′ and R″ together with the N to which they are connected form either
   a. a 5 member heterocycle ring, wherein the heterocycle ring is optionally substituted with OH, or
   b. a 6 member heterocycle ring additionally comprising 1 O heteroatom;
wherein R⁴ can only be alkyl when R⁶ is —OH.

An embodiment of the present invention provides compounds according to formula I as described herein, wherein R⁴ is selected from
i. alkyl,
ii. hydroxyalkyl,
iii. alkoxyalkyl,
iv. cycloalkylalkyl substituted with hydroxyl,
v. a 4-membered heterocycle comprising a single O heteroatom,
vi. (CH₂)ₙC(O)OH, (CH₂)ₙC(O)OCH₃, or (CH₂)ₙC(O)NHCH₃, wherein n is 3,
vii. heteroarylalkyl, wherein the heteroaryl in heteroarylalkyl is an oxazole heteroaryl,
viii. heteroarylalkyl, wherein the heteroaryl in heteroarylalkyl is a pyrazole,
ix. —(CH₂)ₙS(O)₂CH₃ or (CH₂)ₙCN wherein n is 3,
x. —(CH₂)ₙC(O)NR′R″, wherein n is 3 and R′ and R″ are both CH₃, R′ is CH₃ and R″ is a hydroxylalkyl, or R′ and R″ together with the N to which they are connected form either
   a. a 5 member heterocycle ring, wherein the heterocycle ring is optionally substituted with OH, or
   b. a 6 member heterocycle ring additionally comprising 1 O heteroatom;
wherein R⁴ can only be alkyl when R⁶ is —OH.

An embodiment of the present invention provides compounds according to formula I as described herein, wherein R⁴ is selected from
i. alkyl,
ii. hydroxyalkyl,
iii. alkoxyalkyl,
iv. cycloalkyl substituted with hydroxy or alkoxy,
v. cycloalkylalkyl substituted with hydroxyl, alkoxy, or —COOH,
vi. a 4-6 membered heterocycle comprising a single O heteroatom,
vii. (CH₂)ₙC(O)OH, (CH₂)ₙC(O)OCH₃, or (CH₂)ₙC(O)NHCH₃, wherein n is 2 or 3,
viii. heteroarylalkyl, wherein the heteroaryl in heteroarylalkyl is an oxazole heteroaryl.
wherein R⁴ can only be alkyl when R⁶ is —OH.

An embodiment of the present invention provides compounds according to formula I as described herein, wherein R⁴ is selected from
i. alkyl,
ii. hydroxyalkyl,
iii. alkoxyalkyl,
iv. cycloalkylalkyl substituted with hydroxy,
v. a 4-membered heterocycle comprising a single O heteroatom,
vi. (CH₂)ₙC(O)OH, (CH₂)ₙC(O)OCH₃, or (CH₂)ₙC(O)NHCH₃, wherein n is 3,
vii. heteroarylalkyl, wherein the heteroaryl in heteroarylalkyl is an oxazole heteroaryl;
wherein R⁴ can only be alkyl when R⁶ is —OH.

An embodiment of the present invention provides compounds according to formula I as described herein, wherein R⁶ is H.

An embodiment of the present invention provides compounds according to formula I, wherein
R¹ and R⁵, and the atoms to which they are bonded, form either
   i. a 4-6 membered heterocycle ring comprising a single O heteroatom, or
   ii. a 4-5 membered cycloalkyl ring;
R² and R³ are selected from H and alkyl, wherein only one of R² or R³ can be H or alkyl;
R⁴ is selected from
   i. alkoxyalkyl,
   ii. cycloalkyl substituted with hydroxy or alkoxy,
   iii. cycloalkylalkyl substituted with hydroxy or alkoxy, and
   iv. a 4-6 membered heterocycle comprising a single O heteroatom;
R⁶ is H or —OH;
and pharmaceutically acceptable salts.

An embodiment of the present invention provides compounds according to formula I, wherein
R¹ and R⁵, and the atoms to which they are bonded, form either
   i. a 4-6 membered heterocycle ring comprising a single O heteroatom, or
   ii. a 4-5 membered cycloalkyl ring;
R² and R³ are selected from H and alkyl, wherein only one of R² or R³ can be H or alkyl;
R⁴ is selected from
   i. cycloalkyl substituted with hydroxy or alkoxy,
   ii. cycloalkylalkyl substituted with hydroxy or alkoxy, and
   iii. a 4-6 membered heterocycle comprising a single O heteroatom;
R⁶ is H or —OH;
and pharmaceutically acceptable salts.

An embodiment of the present invention provides compounds according to formula I, wherein
R¹ and R⁵, and the atoms to which they are bonded, form either
   i. a 4-6 membered heterocycle ring comprising a single O heteroatom, or
   ii. a 4-5 membered cycloalkyl ring;
R² and R³ are selected from H and alkyl, wherein only one of R² or R³ can be H or alkyl;
R⁴ is selected from
   i. cycloalkyl substituted with hydroxy or alkoxy, and
   ii. cycloalkylalkyl substituted with hydroxy or alkoxy;
R⁶ is H or —OH;
and pharmaceutically acceptable salts.

An embodiment of the present invention provides compounds according to formula I, wherein
R¹ and R⁵, and the atoms to which they are bonded, form a 4-6 membered heterocycle ring comprising a single O heteroatom;
R² and R³ are selected from H and alkyl, wherein only one of R² or R³ can be H or alkyl;
R⁴ is selected from
   i. cycloalkyl substituted with hydroxy, and
   ii. cycloalkylalkyl substituted with hydroxy;
R⁶ is H or —OH;
and pharmaceutically acceptable salts.

An embodiment of the present invention provides compounds according to formula I, wherein
R¹ and R⁵, and the atoms to which they are bonded, form a 4-6 membered heterocycle ring comprising a single O heteroatom;
R² is H and R³ is alkyl;
R⁴ is selected from
   i. cycloalkyl substituted with hydroxy, and
   ii. cycloalkylalkyl substituted with hydroxy;
R⁶ is H or —OH;
and pharmaceutically acceptable salts.

An embodiment of the present invention provides compounds according to formula I, wherein
R¹ and R⁵, and the atoms to which they are bonded, form a 4-6 membered heterocycle ring comprising a single O heteroatom;
R² is H and R³ is alkyl;
R⁴ is selected from
   i. cycloalkyl substituted with hydroxy, and
   ii. cycloalkylalkyl substituted with hydroxy;
R⁶ is H;
and pharmaceutically acceptable salts.

An embodiment of the present invention provides compounds according to formula I, wherein
R¹ and R⁵, and the atoms to which they are bonded, form a 5 membered heterocycle ring comprising a single O heteroatom;
R² is H and R³ is alkyl;
R⁴ is selected from
   i. alkyl,
   ii. hydroxyalkyl,
   iii. alkoxyalkyl,
   iv. cycloalkylalkyl substituted with hydroxy,
   v. a 4-membered heterocycle comprising a single O heteroatom,
   vi. (CH₂)ₙC(O)OH, (CH₂)ₙC(O)OCH₃, or (CH₂)ₙC(O)NHCH₃, wherein n is 3,
   vii. heteroarylalkyl, wherein the heteroaryl in heteroarylalkyl is an oxazole heteroaryl;
R⁶ is H or —OH,
   wherein R⁴ can only be alkyl when R⁶ is —OH;
and pharmaceutically acceptable salts.

An embodiment of the present invention provides compounds according to formula I, wherein
R¹ and R⁵, and the atoms to which they are bonded, form a 5 membered heterocycle ring comprising a single O heteroatom;
R² is H and R³ is methyl;
R⁴ is selected from
   i. alkyl,
   ii. hydroxyalkyl,
   iii. alkoxyalkyl,
   iv. cycloalkylalkyl substituted with hydroxy,
   v. a 4-membered heterocycle comprising a single O heteroatom,
   vi. (CH₂)ₙC(O)OH, (CH₂)ₙC(O)OCH₃, or (CH₂)ₙC(O)NHCH₃, wherein n is 3,
   vii. heteroarylalkyl, wherein the heteroaryl in heteroarylalkyl is an oxazole heteroaryl,
   viii. heteroarylalkyl, wherein the heteroaryl in heteroarylalkyl is a pyrazole;
   ix. —(CH₂)ₙS(O)₂CH₃ or (CH₂)ₙCN wherein n is 3,
   x. —(CH₂)ₙC(O)NR′R″, wherein n is 3 and R′ and R″ are both CH₃, R′ is CH₃ and R″ is a hydroxylalkyl, or R′ and R″ together with the N to which they are connected form either
      a. a 5 member heterocycle ring, wherein the heterocycle ring is optionally substituted with OH, or
      b. a 6 member heterocycle ring additionally comprising 1 O heteroatom;
R⁶ is H or —OH,
   wherein R⁴ can only be alkyl when R⁶ is —OH;
and pharmaceutically acceptable salts.

A particular example of compounds of formula I as described herein is 5-[3-[[(3R)-1-(2-Hydroxyethyl)-3-piperidyl]amino]-5-methyl-1,2,4-triazin-6-yl]-2,3-dihydrobenzofuran-4-ol or pharmaceutically acceptable salts thereof.

Other particular examples of compounds of formula I as described herein are selected from
5-[5-Methyl-3-[[rac-(3R)-1-(2-hydroxypropyl)-3-piperidyl]amino]-1,2,4-triazin-6-yl]-2,3-dihydrobenzofuran-4-ol;
5-[3-[[(3R)-1-(3-Hydroxypropyl)-3-piperidyl]amino]-5-methyl-1,2,4-triazin-6-yl]-2,3-dihydrobenzofuran-4-ol;
5-[3-[[(3R)-1-[(1-Hydroxycyclopropyl)methyl]-3-piperidyl]amino]-5-methyl-1,2,4-triazin-6-yl]-2,3-dihydrobenzofuran-4-ol;
5-[3-[[(3R)-1-(2-Methoxyethyl)-3-piperidyl]amino]-5-methyl-1,2,4-triazin-6-yl]-2,3-dihydrobenzofuran-4-ol; formic acid salt;
5-[3-[[(3R)-1-(2-Methoxyethyl)-3-piperidyl]amino]-5-methyl-1,2,4-triazin-6-yl]-2,3-dihydrobenzofuran-4-ol;
(3S,5R)-1-Ethyl-5-[[6-(4-hydroxy-2,3-dihydrobenzofuran-5-yl)-5-methyl-1,2,4-triazin-3-yl]amino]piperidin-3-ol;
5-[5-Methyl-3-[[rac-(3R)-1-(2-methoxypropyl)-3-piperidyl]amino]-1,2,4-triazin-6-yl]-2,3-dihydrobenzofuran-4-ol;
5-[5-Methyl-3-[[(3R)-1-(oxetan-3-yl)-3-piperidyl]amino]-1,2,4-triazin-6-yl]-2,3-dihydrobenzofuran-4-ol;
4-[(3R)-3-[[6-(4-hydroxy-2,3-dihydrobenzofuran-5-yl)-5-methyl-1,2,4-triazin-3-yl]amino]-1-piperidyl]butanoic acid;
4-[(3R)-3-[[6-(4-hydroxy-2,3-dihydrobenzofuran-5-yl)-5-methyl-1,2,4-triazin-3-yl]amino]-1-piperidyl];
Methyl 4-[(3R)-3-[[6-(4-hydroxy-2,3-dihydrobenzofuran-5-yl)-5-methyl-1,2,4-triazin-3-yl]amino]-1-piperidyl]butanoate;
4-[(3R)-3-[[6-(4-hydroxy-2,3-dihydrobenzofuran-5-yl)-5-methyl-1,2,4-triazin-3-yl]amino]-1-piperidyl]-N-methyl-butanamide;
5-[5-methyl-3-[[(3R)-1-(2-oxazol-2-ylethyl)-3-piperidyl]amino]-1,2,4-triazin-6-yl]-2,3-dihydrobenzofuran-4-ol;
and pharmaceutically acceptable salts thereof.

Other particular examples of compounds of formula I as described herein are selected from
4-[(3R)-3-[[6-(4-hydroxy-2,3-dihydrobenzofuran-5-yl)-5-methyl-1,2,4-triazin-3-yl]amino]-1-piperidyl]-N,N-dimethyl-butanamide;
4-[(3R)-3-[[6-(4-hydroxy-2,3-dihydrobenzofuran-5-yl)-5-methyl-1,2,4-triazin-3-yl]amino]-1-piperidyl]-1-pyrrolidin-1-yl-butan-1-one;
4-[(3R)-3-[[6-(4-hydroxy-2,3-dihydrobenzofuran-5-yl)-5-methyl-1,2,4-triazin-3-yl]amino]-1-piperidyl]-N-(2-hydroxyethyl)-N-methyl-butanamide;
1-(3-hydroxypyrrolidin-1-yl)-4-[rac-(3R)-3-[[6-(4-hydroxy-2,3-dihydrobenzofuran-5-yl)-5-methyl-1,2,4-triazin-3-yl]amino]-1-piperidyl]butan-1-one; formic acid;
1-(3-hydroxypyrrolidin-1-yl)-4-[rac-(3R)-3-[[6-(4-hydroxy-2,3-dihydrobenzofuran-5-yl)-5-methyl-1,2,4-triazin-3-yl]amino]-1-piperidyl]butan-1-one;
4-[(3R)-3-[[6-(4-hydroxy-2,3-dihydrobenzofuran-5-yl)-5-methyl-1,2,4-triazin-3-yl]amino]-1-piperidyl]-1-morpholino-butan-1-one;
5-[5-methyl-3-[[(3R)-1-(2-pyrazol-1-ylethyl)-3-piperidyl]amino]-1,2,4-triazin-6-yl]-2,3-dihydrobenzofuran-4-ol;
5-[5-methyl-3-[[(3R)-1-[3-(1H-tetrazol-5-yl)propyl]-3-piperidyl]amino]-1,2,4-triazin-6-yl]-2,3-dihydrobenzofuran-4-ol; formic acid;
5-[5-methyl-3-[[(3R)-1-[3-(1H-tetrazol-5-yl)propyl]-3-piperidyl]amino]-1,2,4-triazin-6-yl]-2,3-dihydrobenzofuran-4-ol;
4-[(3R)-3-[[6-(4-hydroxy-2,3-dihydrobenzofuran-5-yl)-5-methyl-1,2,4-triazin-3-yl]amino]-1-piperidyl]butanenitrile;
5-[5-methyl-3-[[(3R)-1-(3-oxazol-2-ylpropyl)-3-piperidyl]amino]-1,2,4-triazin-6-yl]-2,3-dihydrobenzofuran-4-ol;
4-[(3R)-3-[[6-(4-hydroxy-2,3-dihydrobenzofuran-5-yl)-5-methyl-1,2,4-triazin-3-yl]amino]-1-piperidyl]-N,N-dimethyl-butanamide; formic acid”
4-[(3R)-3-[[6-(4-hydroxy-2,3-dihydrobenzofuran-5-yl)-5-methyl-1,2,4-triazin-3-yl]amino]-1-piperidyl]-N,N-dimethyl-butanamide; formic acid;
5-[5-methyl-3-[[(3R)-1-[2-(1H-tetrazol-5-yl)ethyl]-3-piperidyl]amino]-1,2,4-triazin-6-yl]-2,3-dihydrobenzofuran-4-ol;
5-[5-methyl-3-[[(3R)-1-(3-methylsulfonylpropyl)-3-piperidyl]amino]-1,2,4-triazin-6-yl]-2,3-dihydrobenzofuran-4-ol;
and pharmaceutically acceptable salts thereof.

A more preferred example of a compound of formula I as described herein is 4-[(3R)-3-[[6-(4-hydroxy-2,3-dihydrobenzofuran-5-yl)-5-methyl-1,2,4-triazin-3-yl]amino]-1-piperidyl]butanoic acid or pharmaceutically acceptable salts thereof.

Other more preferred examples of a compound of formula I as described herein are selected from
5-[5-methyl-3-[[(3R)-1-[3-(1H-tetrazol-5-yl)propyl]-3-piperidyl]amino]-1,2,4-triazin-6-yl]-2,3-dihydrobenzofuran-4-ol;
4-[(3R)-3-[[6-(4-hydroxy-2,3-dihydrobenzofuran-5-yl)-5-methyl-1,2,4-triazin-3-yl]amino]-1-piperidyl]-N,N-dimethyl-butanamide;
4-[(3R)-3-[[6-(4-hydroxy-2,3-dihydrobenzofuran-5-yl)-5-methyl-1,2,4-triazin-3-yl]amino]-1-piperidyl]-1-pyrrolidin-1-yl-butan-1-one;
and pharmaceutically acceptable salts thereof.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a metabolic disease;
(vi) a cardiovascular disease;
(vii) a respiratory disease;
(viii) a liver disease;
(ix) a renal disease;
(x) an ocular disease;
(xi) a skin disease;
(xii) a lymphatic condition;
(xiii) graft versus host disease;
(xiv) allodynia;
(xv) a condition associated with diabetes; and
(xvi) any disease where an individual has been determined to carry a germline or somatic non-silent mutation in NLRP3

In another embodiment, the disease, disorder or condition is selected from:
(i) cancer;
(ii) an infection;
(iv) a cardiovascular disease;
(v) a liver disease;
(vi) a respiratory disease;
(vi) an ocular disease; and
(vii) a skin disease.

In another embodiment, the disease, disorder or condition is selected from:
(i) gout; and
(ii) arthritis.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia; (vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(x) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xi) a lymphatic condition such as Castleman's disease;
(xii) a condition of, or involving, the immune system, such as hyper IgE syndrome, lepromatous leprosy, familial hemophagocytic lymphohistiocytosis, or graft versus host disease;
(xiii) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xiv) a cancer, including those cancers listed above;
(xv) a burn, wound, trauma, haemorrhage or stroke;
(xvi) radiation exposure;
(xvii) a metabolic disease such as type 2 diabetes (T2D), atherosclerosis, obesity, gout or pseudo-gout; and/or
(xviii) pain such as inflammatory hyperalgesia, pelvic pain, allodynia, neuropathic pain, or cancer-induced bone pain.

An embodiment of the present invention is a compound according to formula I as described herein for the treatment or prophylaxis of a disease, disorder or condition selected from:
(i) inflammation;
(ii) an auto-immune disease;
(iii) cancer;
(iv) an infection;
(v) a metabolic disease;
(vi) a cardiovascular disease;
(vii) a respiratory disease;
(viii) a liver disease;
(ix) a renal disease;
(x) an ocular disease;
(xi) a skin disease;
(xii) a lymphatic condition;
(xiii) a psychological disorder;
(xiv) graft versus host disease;
(xv) allodynia;
(xvi) a condition associated with diabetes; and
(xvii) any disease where an individual has been determined to carry a germline or somatic non-silent mutation in NLRP3.

An embodiment of the present invention is the use of a compound according to formula I as described herein in the treatment or prophylaxis of a disease, disorder or condition, wherein the disease, disorder or condition is responsive to NLRP3 inhibition.

An embodiment of the present invention is the use a compound according to formula I as described herein for use in the treatment or prophylaxis of a disease, disorder or condition selected from Asthma and COPD.

An embodiment of the present invention is the use a compound according to formula I as described herein for use in the treatment or prophylaxis of a cardiovascular disease, disorder or condition.

An embodiment of the present invention is the use a compound according to formula I as described herein for use in the treatment or prophylaxis of a disease, disorder or condition selected from Cryopyrin-associated periodic syndromes.

An embodiment of the present invention is a compound according to formula I as described herein for the treatment or prophylaxis of a disease, disorder or condition selected from Asthma and COPD.

An embodiment of the present invention is a compound according to formula I as described herein for the treatment or prophylaxis of a cardiovascular disease, disorder or condition.

An embodiment of the present invention is a compound according to formula I as described herein for the treatment or prophylaxis of a disease, disorder or condition selected from Cryopyrin-associated periodic syndromes.

An embodiment of the present invention is the use of a compound according to formula I as described herein for the preparation of a medicament for the treatment or prophylaxis of a disease, disorder or condition selected from Asthma and COPD.

An embodiment of the present invention is the use of a compound according to formula I as described herein for the preparation of a medicament for the treatment or prophylaxis of a cardiovascular disease, disorder or condition.

An embodiment of the present invention is the use of a compound according to formula I as described herein for the preparation of a medicament for the treatment or prophylaxis of a disease, disorder or condition selected from Cryopyrin-associated periodic syndromes.

An embodiment of the present invention is a method of treatment or prophylaxis of a disease, disorder or condition selected from Asthma and COPD, which method comprises administering an effective amount of a compound according to formula I as described herein.

An embodiment of the present invention is a method of treatment or prophylaxis of a cardiovascular disease, disorder or condition, which method comprises administering an effective amount of a compound according to formula I as described herein.

An embodiment of the present invention is a method of treatment or prophylaxis of a disease, disorder or condition selected from Cryopyrin-associated periodic syndromes, which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (~10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
Add 5 μl compound (8 points half-log dilution, with 10 μM top dose) or vehicle (DMSO 0.1% FAC) to the appropriate wells
Incubate for 3 hours at 37° C., 5% CO₂
Add 5 μl nigericin (Sigma #N7143) (FAC 5 μM) to all wells
Incubate for 1 hr at 37° C., 5% CO₂
At the end of the incubation period, spin plates at 300×g for 3 mins and remove supernatant
Then add 50p of resazurin (Sigma #R⁷⁰¹⁷) (FAC 100 μM resazurin in RPMI medium without FBS) and incubate plates for a further 1-2 hours at 37° C. and 5% CO₂
Plates were read in an Envision reader at Ex 560 nm and Em 590 nm
IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.
Human whole blood in Li-heparin tubes was obtained from healthy donors from a volunteer donor panel.
Plate out 80 μl of whole blood containing 1 μg/ml of LPS in 96-well, clear bottom cell culture plate (Corning #3585)
Add 10 μl compound (8 points half-log dilution with 10 μM top dose) or vehicle (DMSO 0.1% FAC) to the appropriate wells
Incubate for 3 hours at 37° C., 5% CO₂
Add 10 μl nigericin (Sigma #N7143) (10 μM FAC) to all wells
Incubate for 1 hr at 37° C., 5% CO₂
At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
IL-1β was measured according to the manufacturer protocol (Perkin Elmer-AlphaLisa IL-1 Kit AL220F-5000)
IC₅₀ data is fitted to a non-linear regression equation (log inhibitor vs response-variable slope 4-parameters)

The results of the human whole blood assay are summarised in Table 1 below as HWB IC₅₀.

### Microsomal Stability:

Incubations of test compounds at 1 μM in microsomes (0.5 mg/mL) plus cofactor NADPH are performed in 96 well plates at 37° C. on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

### Assay Descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures; pooled (n=5 for male and n=5 for female for human)] with stromal mouse fibroblasts (negative control; pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 mg/mL hydrocortisone and 0.004 mg/mL insulin, to a final suspension density of 1×106 cells/mL. The incubation was performed fully automatically with Liquid Handling System (Tecan) equipped with a CO2 incubator with an orbital shaker. After the addition of a test compound at e.g. 1 μM to the wells (1×105 cells/well), the 96-well hepatocyte suspension culture plates are incubated in a 5% CO2 at 37° C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO2 atmosphere and 37° C.). The incubation media in human HepatoPac® is identical with that in suspended hepatocytes. At defined time points (2, 18, 26, 48, 72 and 96 h), whole wells are quenched with ice-cold acetonitrile containing an internal standard.

Samples are then centrifuged appropriately and the supernatant analyzed by LC-MS/MS. The incubation is conducted in n=1 or 2.

hERG screening assay

In the drug development process of small molecules, one of the most frequent adverse side effects, leading to the failure of drugs, is the cardiac arrhythmias. Such failure is often related to the capacity of the drug to inhibit the human ether-á-go-go-related gene (hERG) cardiac potassium channel. Having no or low inhibition of the hERG cardiac potassium channel is therefore considered as beneficial.

### Cells

The CHO crelox hERG cell line (ATCC reference Nr. PTA-6812, female Chinese hamster cells) was generated and validated at Roche. Ready-to-use frozen instant CHO-hERG cells were cryopreserved at Evotec (Germany) and used directly in the experiments.

### Experimental Solutions

The extracellular solution contains (in mM): NaCl 150; KCl 4; CaCl₂ 1; MgCl₂ 1; HEPES 10; pH 7.2-7.4 with NaOH, osmolarity 290-330 mOsm. The internal solution contains (in mM): KCl, 10; KF, 100; NaCl, 10; HEPES, 10; EGTA, 20; pH=7.0-7.4 with KOH, osmolarity 260-300 mOsm.

### Electrophysiology

The effects of a compound on hERG K+-currents parameters will be evaluated at 2 concentrations in at least 4 cells.

The hERG test is performed using automated patch clamp system SynchroPatch® 384 (Nanion Technologies GmbH, Germany). K+ currents are measured with the patch-voltage-clamp technique in the whole-cell configuration at 35-37° C.

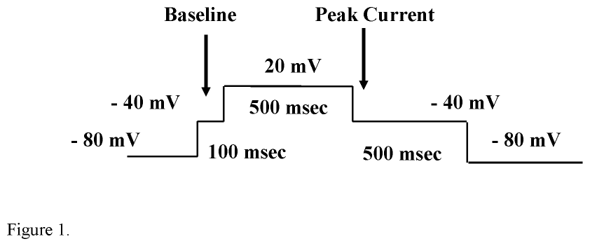

Cells were held at a resting voltage of −80 mV and they were stimulated by a voltage pattern shown in FIG. 1 (pulse pattern used to elicit outward K⁺ current at 35-37° C.) to activate hERG channels and conduct outward IKhERG current, at a stimulation frequency of 0.1 Hz (6 bpm)

### Data Analysis

The amplitudes of IKhERG were recorded in each concentration of drug and they were compared to the vehicle control values (taken as 100%) to define fractional blocks. The concentration-response data were fitted with the following relationship:

$I ( C ) = \frac{100}{1 + {( C / IC 50 )}^{h}}$

| | |
|---|---|
| where | C is the concentration, |
| | IC₅₀ is the concentration producing 50% block |
| | h is the Hill coefficient. |

Concentration-response curves were fitted by non-linear regression analysis using EworkBook suite (ID Business Solutions Ltd, UK). Data fit was done with the 4 Parameter Logistic Model (fit=(A+(B/(1+((x/C)\hat{ }D)))), where A=0 and B=100).

The results of the hERG assay are summarised in Table 2 below as hERG IC₂₀.

### Transcellular P-gp Assay:

The general assay uses transfected LLC-PK1 cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer. The tightness of the cell monolayer and functional activity of P-gp are confirmed by addition of a cell-impermeable marker, Lucifer yellow, and a reference P-gp substrate, edoxaban, respectively. J. Pharmacol. Exp Ther., 2021, 376, 322-329.

### PAMPA:

PAMPA (Parallel Artificial Membrane Permeability Assay) is a first line permeability screen for drug candidates. The PAMPA assay mimics the transcellular absorption conditions using an artificial phospholipid membrane. This assay determines a permeability value that can be used for compound optimization and ranking purposes as well as input parameters for in silico models to predict intestinal absorption.

The donor concentration is measured at t-start (reference) and compared with the donor and acceptor concentration after a certain time (t-end) to calculate the extent of passage of the compound through the membrane.

### Bacterial Reverse Mutation Test (AMES):

The testing of compounds is conducted as outlined in this guideline: Test No. 471: Bacterial Reverse Mutation Test|OECD Guidelines for the Testing of Chemicals, Section 4: Health Effects|OECD iLibrary (oecd-ilibrary.org)

### Bacteria Culture:

The bacterial strains used are TA98, TA100, TA1535, TA97a and TA102. Batches of each strain, are maintained as frozen stocks. Vials are thawed and used to inoculate cultures in nutrient broth. The cultures are placed in an incubator set to 37° C. with agitation for approximately 10 hours to provide a working culture of at least 108 cells per mL.

To ensure cultures are at the appropriate phase of growth and culture density, a sample is taken from each culture at the end of the incubation period and assessed for culture density by either viability plating or OD650 assessment.

### Treatment:

3 replicates per concentration of compound and positive controls and 6 replicates per vehicle controls are included.

Formulations are prepared using DMSO to allow maximum exposure up to the solubility limit or 1000 μg/well for a freely soluble test article. This concentration is equivalent to 5000 μg/plate as used in the usual plate incorporation Ames assay.

Concentrations are usually separated by half-log intervals in a single experiment. For soluble compounds, concentrations will be 0, 3.2, 10, 32, 100, 320, 1000 μg/well.

Positive controls used are:

Platings will be achieved by the following sequence of additions to 400 μL supplemented molten agar at 45±1° C.:
20 μL of bacterial culture
20 μL of test article solution/vehicle control/positive control
100 μL of 10% S-9 mix or buffer solution
   followed by rapid mixing and pouring onto mutation plates (wells).

When set, the plates will be inverted and incubated protected from light for 2 to 3 days in an incubator set to 37° C.

### Toxicity:

Toxicity is detected by the following parameters:
Diminution of background lawn
Marked reduction in revertants compared to the concurrent vehicle controls
Reduction in mutagenic response.

### Scoring:

Scoring of bacteria colonies is performed manually or electronically using automated colony counter.

### In Vitro Mammalian Cell Micronucleus Test:

The testing of compounds is conducted as outlined in this guideline: Test No. 487: In Vitro Mammalian Cell Micronucleus Test|OECD Guidelines for the Testing of Chemicals, Section 4: Health Effects|OECD iLibrary (oecd-ilibrary.org)

### Cell Culture:

Cultures are maintained in tissue culture flasks containing HEPES-buffered RPMI 1640 medium with GlutaMAX-1 including 10% (v/v) heat inactivated foetal calf serum, 100 Units/mL/100 μg/mL penicillin/streptomycin in a humidified incubator set to 37° C., 5% (v/v) CO2 in air. Cells will be subcultured at low to medium density at least once prior to treatment. On the day prior to treatment, cells will be subcultured at a density of approximately 7×104 cells/mL. Cells will be maintained at 37° C., 5% (v/v) CO2 in air, in a humidified environment prior to treatment.

### Treatment:

Cultured human lymphoblastoid TK6 cells will be exposed to the compound for 3 hours in the presence of S-9, followed by a recovery period of 24 hours. In addition, a continuous 27 hour treatment in the absence of S-9 will be included as a number of chemicals have been reported as only exerting positive effects following prolonged treatment. This is equivalent to approximately 1.5-2.0 times the average generation time of the TK6 cells used in this laboratory (cell cycle approximately 15 hours). All cultures will be sampled 27 hours after the beginning of treatment. Dilutions will be prepared in DMSO that allow maximum exposure up to the solubility limit, 1 mM or 500 μg/mL, whichever is lower.

Normally, at least 12 concentrations separated by 0.7-fold intervals, ranging down from the upper limit (for soluble compounds with a MW≥500, concentrations will be 9.887, 14.12, 20.18, 28.82, 41.18, 58.82, 84.04, 120.1, 171.5, 245, 350 and 500 μg/mL). The final concentration of DMSO will be 1% v/v. Positive controls are Noscapine in the absence of S-9 and Cyclophosphamide in the presence of S-9. 2 replicates per concentration of compound and multiple concurrent vehicle and positive controls will be included per treatment in 96-well plates and incubated for the treatment time at 37° C., 5% (v/v) Co₂. 3 hour treatment cultures will be washed once and reincubated with fresh medium for 24 hours.

### Harvesting:

At the defined sampling time an aliquot of cell suspension from designated cultures will be taken for determination of cell number by using a Coulter Counter. Cultures designated for analysis will be centrifuged at approximately 200 g, 5 minutes. Cells will be resuspended in 0.075 M KCl prior to fixation in fresh, cold methanol/glacial acetic acid (7:1 v/v). Fixed cells will be stored in fixative at 2-8° C. prior to slide preparation.

Slides will be air-dried prior to staining by immersion in 12.5 μg/mL Acridine Orange in phosphate buffered saline (PBS), pH 6.8 for approximately 10 minutes, following by a wash with PBS (with agitation) for a few seconds.

### Cytotoxicity Readout and Selection of Concentrations:

Toxicity is expressed as Population Doubling (PD) relative to vehicle controls. PD will be calculated for each concentration as follows:

$$P D = [ log ( N / {X}_{0} ) ] / log 2$$

Where N=mean final cell count/culture at each concentration
   X₀=starting (baseline) count The highest concentration for micronucleus analysis should either not exceed (approximately) 50% cytotoxicity, be the highest concentration tested, or, be the lowest precipitating concentration observed by eye at the end of the treatment incubation period. Slides from the highest selected concentration and at least two lower concentrations will be analysed, such that a range of cytotoxicity from maximum to little or none is covered, where appropriate. A minimum of 1000 mononucleate cells from each culture (2000 per concentration) will be analysed for micronuclei.

### Evaluation Criteria:

The compound will be considered to induce clastogenic and/or aneugenic events if:
A statistically significant increase in the frequency of MNMON cells at one or more concentrations is observed.
The incidence of cells with micronuclei at such a concentration exceeds the normal range in both replicates.
A concentration-related increase in the proportion of cells with micronuclei is observed (positive trend test).

The compound will be considered positive in this assay if all of the above criteria are met. The compound will be considered negative in this assay if none of the above criteria are met. Results which only partially satisfy the above criteria will be dealt with on a case-by-case basis, but in the context of the screening study, will be concluded as either positive, negative or equivocal. Evidence of a concentration-related effect is considered useful but not essential in the evaluation of a positive result. Biological relevance will be taken into account, for example consistency of response within and between concentrations and (if applicable) between experiments, or effects occurring only at very toxic concentrations.$

### Pharmacokinetics Profile of Test Substances in Minipigs:

The pharmacokinetics of the test substance was determined in minipigs following intravenous and oral administration. The experimental design consisted of three male minipigs, of which each animal received a single intravenous bolus dose, and a single oral doses with the test item. Intravenous doses were administered at a nominal dose volume of 1 mL/kg. Oral doses were administered by gavage at a nominal dose volume of 5 mL/kg. There was a washout period of at least 7 days between last sampling occasion and the next dosing occasion to the same animal. The content of all formulations was within the desired range of 85 to 115% of the nominal content. Following dosing, samples of blood (1 mL) were withdrawn from the saphenous (via cannula) or jugular vein of each animal at pre-dose, 5, 15, 30 min, 1, 2, 4, 8, 24 hours post-dose after IV dosing and at pre-dose, 15, 30 min, 1, 2, 4, 6, 8, 24, 48 hours post-dose after oral dosing. At all time points, the haematocrit was determined. The blood:plasma partitioning factor was determined at the 2 and 4 hour time point, and urine was collected as a single sample for 24 hours after dose administration. Blood samples (nominally 1 mL) were withdrawn from the saphenous (via cannula) or jugular vein of each animal into polypropylene tubes containing K2EDTAanticoagulant and was centrifuged (1500 g, 10 min. 4° C.) to prepare plasma for analysis. Residual blood cells were discarded. Plasma vials were capped and stored on wet-ice for no longer than 60 minutes before being transferred to <−50° C. storage (nominally −80° C.) prior to analysis with a specific LC-MS method.

### Toxicity Assessment of Test Substance in Minipig

The maximum tolerated dose (MTD) of the test item is determined following once daily oral (gavage) administration to the minipig. The toxicity of repeated daily administration for 14 days is then assessed. In addition, the toxicokinetic profile of the test item is characterized. Sufficient purpose-bred Gottingen minipigs are obtained from Ellegaard Gottingen, Dalmose, Denmark (Animals: 2 to 3 month age range and in a 4 to 6 kg weight range). At start of dosing animals are 4 to 5 months old and in a 6 to 9.5 kg weight range. A dose volume of 10 mL/kg is used. Individual dose volumes are calculated from the most recent body weights for each animal to target dose levels of 30, 100 and 300 mg/kg/day or others depending non MTD results. Blood samples are taken on day 1 and day 14 for the determination of drug concentration in plasma and derived toxicokinetic parameters. Animals are not fed on the day of scheduled necropsy. Each animal is anaesthetized via intramuscular injection of a Zoletil mix then killed by exsanguination. All tissues are preserved in the appropriate fixative/s. Further analysis includes food consumption, body weight, clinical pathology, and full histopathological examination of target organs.

TABLE 1
NLRP3 inhibitory activity

| | THP-1 pyroptosis | Human whole blood IL-1β |
|---|---|---|
| | Assay IC₅₀ | Assay IC₅₀ |
| Example No. | (nM) | (nM) |
| 1 | 2.3 | 5.0 |
| 2 | 13 | 11 |
| 3 | 16 | 14 |
| 4 | 17 | 33 |
| 5 | 19 | 15 |
| 6 | 28 | 23 |
| 7 | 62 | 40 |
| 8 | 120 | n.d. |
| 9 | 4.2 | 10.1 |
| 10 | 27 | 40 |
| 11 | 16 | 38 |
| 12 | 53 | 30 |
| 13 | 5 | 20 |
| 14 | 76 | n.d. |
| 15 | 58 | 33 |
| 16 | 40 | 46 |
| 17 | 35 | 76 |
| 18 | 23 | 23 |
| 19 | 20 | 26 |
| 20 | 16 | 18 |
| 21 | 20 | 166 |
| 22 | 58 | 54 |

TABLE 2
hERG inhibition assay

| | hERG assay |
|---|---|
| Example No. | IC₂₀ (μM) |
| 1 | >10 |
| 6 | >10 |
| 9 | >10 |
| 10 | 2.9 |
| 12 | >10 |
| 18 | >10 |

TABLE 3
Transcellular human P-gp Assay:

| Example | Permeability | |
|---|---|---|
| No. | [nm/sec] | ER |
| 1 | 197 | 1.3 |
| 6 | 180 | 1.8 |
| 9 | 4 | 1.0 |
| 10 | 122 | 8.5 |
| 12 | 227 | 4.0 |
| 17 | 314 | 1.3 |
| 18 | 1.6 | 1.6 |
| 20 | 264 | 1.2 |
| 22 | 258 | 3 |

* ER = efflux ratio

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization.

### Experimental Methods

### Abbreviations

| | |
|---|---|
| AcOH | acetic acid |
| ACN | acetonitrile |
| DMSO | dimethyl sulfoxide |
| DCE | dichloroethane |
| DIEA | N,N-diisopropylethylamine |
| DIBAl-H | diisobutylaluminium hydride |
| EA, EtOAc | ethyl acetate |
| h, hrs | hour, hours |
| HPLC | high performance liquid chromatography |
| min(s) | minute(s) |
| NaBH₃CN | sodium cyanoborohydride |
| NaBH(OAc)₃ | sodium triacetoxyborohydride |
| r.t. | room temperature |
| TEA | triethylamine |
| TFA | Trifluoroacetic acid |

### EXAMPLES

All examples and intermediates were prepared under nitrogen atmosphere if not specified otherwise.

### Example 1

### 5-[3-[[(3R)-1-(2-Hydroxyethyl)-3-piperidyl]amino]-5-methyl-1,2,4-triazin-6-yl]-2,3-dihydrobenzofuran-4-ol

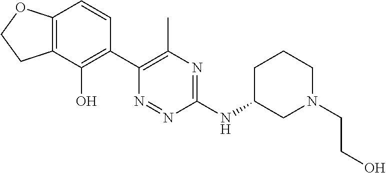 [SMILES: Cc1nc(N[C@@H]2CCCN(CCO)C2)nnc1-c1ccc2c(c1O)CCO2]

### Step A: 4-Benzyloxy-5-bromo-2,3-dihydrobenzofuran

To a solution of 5-bromocoumaran-4-ol (CAS #2279149-27-6, 4.59 g, 20.26 mmol, 1.00 eq) in acetonitrile (40 mL) was added potassium carbonate (5.6 g, 40.51 mmol, 2.00 eq) followed by benzyl bromide (4.89 g, 3.4 mL, 28.57 mmol, 1.41 eq). The reaction mixture was stirred at room temperature for 2 hours. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 220 g, gradient 0% to 10% ethyl acetate in heptane) to afford the title compound (6.17 g, 95% yield) as a colorless oil. LCMS: m/z 305.1/307.0 [M+H]⁺, ESI pos.

### Step B: 2-(4-Benzyloxy-2,3-dihydrobenzofuran-5-yl)-4,4,5,5-tetramethyl-1,3,2-dioxaborolane

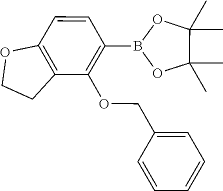 [SMILES: CC1(C)OB(c2ccc3c(c2OCc2ccccc2)CCO3)OC1(C)C]

To a solution of 4-benzyloxy-5-bromo-2,3-dihydrobenzofuran (Example 1, step A) (6.16 g, 19.18 mmol, 1.00 eq) and 2-isopropoxy-4,4,5,5-tetramethyl-1,3,2-dioxaborolane (CAS #61676-62-8, 5.47 g, 6.0 mL, 29.41 mmol, 1.53 eq) in tetrahydrofuran (80 mL) was added dropwise n-butyllithium, 1.6 M solution in hexanes (19 mL, 30.4 mmol, 1.59 eq) within 40 minutes at −76° C. Let stir at −76° C. for 2.5 hours. The reaction mixture was warmed to −60° C., quenched with saturated aq. NH₄Cl-solution at −60° C., warmed to room temperature and then extracted with ethyl acetate and saturated aq. NH₄Cl-solution. The aqueous layer was backextracted with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 120 g, gradient 0% to 10% ethyl acetate in heptane) to afford the title compound (5.78 g, 81% yield) as a colorless oil. LCMS: m/z 353.1 [M+H]⁺, ESI pos.

### Step C: 5-(4,4,5,5-Tetramethyl-1,3,2-dioxaborolan-2-yl)-2,3-dihydrobenzofuran-4-ol

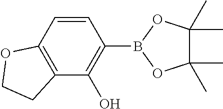 [SMILES: CC1(C)OB(c2ccc3c(c2O)CCO3)OC1(C)C]

A solution of 2-(4-benzoxycoumaran-5-yl)-4,4,5,5-tetramethyl-1,3,2-dioxaborolane (Example 1, step B) (5.77 g, 15.56 mmol, 1.00 eq) in ethyl acetate (70 mL) was three times alternating evacuated and flushed with argon. Palladium on activated charcoal, 10% Pd basis (577 mg, 0.54 mmol, 0.03 eq) was added. The reaction flask was evacuated, flushed with argon, evacuated and flushed with hydrogen. The reaction mixture was stirred under hydrogen atmosphere (balloon) at room temperature for 3 hours. Methanol (10 mL) was added. The reaction flask was three times alternating evacuated and flushed with argon, evacuated and then flushed with hydrogen. The reaction mixture was stirred under hydrogen atmosphere (balloon) at room temperature for 1 hour. The reaction mixture was filtered and rinsed well with ethyl acetate/methanol. The filtrate was concentrated in vacuo to afford the title compound (4.22 g, 98% yield) as an off-white solid, which was used without further purification. LCMS: m/z 263.2 [M+H]⁺, ESI pos.

### Step D: tert-Butyl (3R)-3-[(6-chloro-5-methyl-1,2,4-triazin-3-yl)amino]piperidine-1-carboxylate

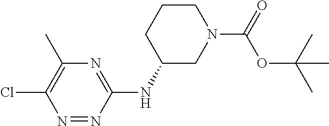 [SMILES: Cc1nc(N[C@@H]2CCCN(C(=O)OC(C)(C)C)C2)nnc1Cl]

To a mixture of 3,6-dichloro-5-methyl-1,2,4-triazine (CAS #132434-82-3, 180 mg, 1.10 mmol, 1.00 eq) and tert-butyl (3R)-3-aminopiperidine-1-carboxylate (CAS #188111-79-7, 330 mg, 1.65 mmol, 1.50 eq) in 1,4-dioxane (3.6 mL) was added N,N-diisopropylethylamine (148 mg, 0.20 mL, 1.15 mmol, 1.04 eq). The reaction mixture was stirred at room temperature for 16 hours. The reaction mixture extracted with dichloromethane and water. The organic layer was washed with brine. The aqueous layers were backextracted twice with dichloromethane. The combined organic layers were dried over sodium sulfate, filtered and concentrated in vacuo. The crude product was adsorbed on ISOLUTE HM-N and purified by flash chromatography (silica gel, 12 g, gradient 0% to 40% ethyl acetate in heptane). All fractions containing product were combined and concentrated in vacuo to afford the title compound (351 mg, 93% yield) as a yellow oil. LCMS: m/z 328.3 [M+H]⁺, ESI pos.

### Step E: 6-Chloro-5-methyl-N-[(3R)-3-piperidyl]-1,2,4-triazin-3-amine

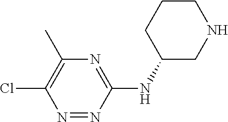

To a solution of tert-butyl (3R)-3-[(6-chloro-5-methyl-1,2,4-triazin-3-yl)amino]piperidine-1-carboxylate (Example 1, step D) (150 mg, 0.43 mmol, 1.00 eq) in dichloromethane (1.4 mL) and methanol (0.70 mL) was added dropwise 4 M HCl in dioxane (1.32 g, 1.1 mL, 4.4 mmol, 10.1 eq). The reaction mixture was stirred at room temperature for 1 hour. The reaction mixture was concentrated in vacuo. The residue was dissolved in dichloromethane/methanol (9:1), basified with saturated aq. NaHCO₃-solution and extracted three times with a mixture of dichloromethane/methanol (9:1). The combined organic layers were dried over sodium sulfate, filtered and concentrated in vacuo to afford the title compound (95 mg, 91% yield) as an orange foam. LCMS: m/z 228.1 [M+H]⁺, ESI pos.

### Step F: 2-[(3R)-3-[(6-Chloro-5-methyl-1,2,4-triazin-3-yl)amino]-1-piperidyl]ethanol

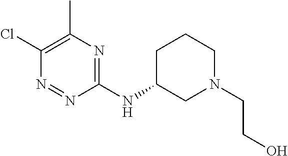 [SMILES: Cc1nc(N[C@@H]2CCCN(CCO)C2)nnc1Cl]

To a solution of 6-chloro-5-methyl-N-[(3R)-3-piperidyl]-1,2,4-triazin-3-amine (Example 1, step E) (95 mg, 0.40 mmol, 1.00 eq) in tetrahydrofuran (1.6 mL) was added 2-iodoethanol (CAS #624-76-0, 84 mg, 0.038 mL, 0.49 mmol, 1.23 eq) followed by N,N-diisopropylethylamine (133 mg, 0.180 mL, 1.03 mmol, 2.60 eq). The reaction mixture was stirred at 40° C. for 16 hours. The reaction mixture was cooled to room temperature, quenched with saturated aq. NaHCO₃-solution and then extracted with ethyl acetate. The aqueous layer was backextracted with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (61 mg, 54% yield) as a light yellow oil. LCMS: m z 272.2 [M+H]⁺, ESI pos.

### Step G: 5-[3-[[(3R)-1-(2-Hydroxyethyl)-3-piperidyl]amino]-5-methyl-1,2,4-triazin-6-yl]-2,3-dihydrobenzofuran-4-ol

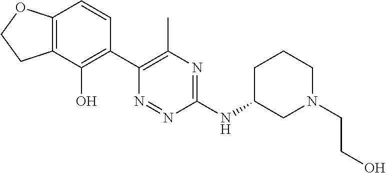 [SMILES: Cc1nc(N[C@@H]2CCCN(CCO)C2)nnc1-c1ccc2c(c1O)CCO2]

A mixture of 2-[(3R)-3-[(6-chloro-5-methyl-1,2,4-triazin-3-yl)amino]-1-piperidyl]ethanol (Example 1, step F) (56 mg, 0.20 mmol, 1.00 eq), 5-(4,4,5,5-tetramethyl-1,3,2-dioxaborolan-2-yl)-2,3-dihydrobenzofuran-4-ol (Example 1, step C) (87 mg, 0.32 mmol, 1.61 eq), cesium carbonate (192 mg, 0.59 mmol, 3.01 eq) and XPhos Pd G3 (25 mg, 0.03 mmol, 0.15 eq) in 1,4-dioxane (1.2 mL) and water (0.30 mL) was flushed with argon and stirred at 100° C. for 2.5 hours. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The residue was adsorbed on ISOLUTE HM-N and purified by flash chromatography (silica gel, 12 g, gradient 0% to 100% (dichloromethane:methanol:NH₄OH 9:1:0.05) in dichloromethane) to afford the title compound (49 mg, 64% yield) as a yellow foam. LCMS: m/z 372.3 [M+H]⁺, ESI pos.

### Example 2

### 5-[5-Methyl-3-[[(3R)-1-(2-hydroxypropyl)-3-piperidyl]amino]-1,2,4-triazin-6-yl]-2,3-dihydrobenzofuran-4-ol

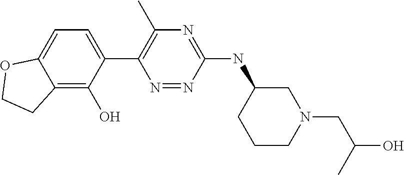 [SMILES: Cc1nc(N[C@@H]2CCCN(CC(C)O)C2)nnc1-c1ccc2c(c1O)CCO2]

### Step A: Methyl 2-benzyloxypropanoate

To a solution of methyl 2-hydroxypropanoate (CAS: 547-64-8; 5.0 g, 48.03 mmol, 1.0 eq) in THF (90 mL) was added NaH (2882 mg, 60% in mineral oil, 72.05 mmol, 1.5 eq) and stirred at 0° C. for ten minutes, then benzyl bromide (CAS: 100-39-0; 5.71 mL, 48.0 mmol, 1.0 eq) was added and the reaction mixture warm room temperature stirred for 1 hour. The mixture was quenched with iced aq. NH₄Cl (150 mL), and extracted with EtOAc (100 mL×2). The organic phase was washed with brine (100 mL×2), dried over anhydrous Na₂SO₄, filtered and concentrated in vacuum. The residue was purified by column chromatography (SiO2, petroleum ether:ethyl acetate, 9:1 to 4:1) to afford the title compound (6.9 g, 74% yield) as colorless oil. ¹H NMR (400 MHz, DMSO-d6) δ[ppm]: 7.35-7.2 (m, 5H), 4.57 (d, 1H), 4.44 (d, 1H), 4.13 (q, 1H), 3.67 (s, 3H), 1.33 (d, 3H).

### Step B: 2-(Benzyloxy)propanal (CAS: 53346-05-7)

To a solution of methyl 2-benzyloxypropanoate (1.0 g, 5.15 mmol, 1.0 eq) in DCM (40 mL) was dropwise added DIBAl-H (7.72 mL, 7.72 mmol, 1.5 eq) over 10 minutes at −78° C. under N₂, and the reaction mixture was stirred for 1 h at −78° C. Upon reaction completion, the mixture was quenched with ice NH₄Cl (100 mL) and extracted with DCM (100 mL×2). The organic phase was washed with brine (100 mL×2), dried over anhydrous Na₂SO₄, filtered and concentrated in vacuum. The residue was purified by column chromatography (SiO₂, petroleum ether:ethyl acetate, 4:1) to afford the title compound (320.0 mg, 38% yield) as colorless oil. ¹H NMR (400 MHz, DMSO-d₆) δ [ppm]: 9.64 (d, 1H), 7.37-7.31 (m, 5H), 4.62-4.55 (m, 2H), 4.52-4.04 (m, 1H), 1.23 (d, 3H).

### Step C: Benzyl N-[(3R)-1-(2-benzyloxypropyl)-3-piperidyl]carbamate

To a mixture of benzyl N-[(3R)-3-piperidyl]carbamate (CAS: 478646-32-1; 228 mg, 0.97 mmol, 0.5 eq), acetic acid (117 mg, 1.95 mmol, 1.0 eq) in DCE (4 mL)/methanol (2 mL) was added 2-benzyloxypropanal (CAS: 53346-05-7; 320.0 mg, 1.95 mmol, 1.0 eq) and stirred for 10 minutes, then NaBH(OAc)₃ (2065 mg, 9.74 mmol, 5.0 eq) was added and stirred at 25° C. for 2 h under N₂. Upon reaction completion, the reaction mixture was concentrated in vacuum. The residue was purified by column chromatography (SiO₂, EA/MeOH: 4/1) to afford the title compound (210 mg, 28% yield) as a colorless oil. LCMS: m/s 383.4[M+H]+, ESI pos.

### Step D: (3R)-1-(2-(Benzyloxy)propyl)piperidin-3-amine

To a solution of benzyl N-[(3R)-1-(2-benzyloxypropyl)-3-piperidyl]carbamate (210.0 mg, 0.55 mmol, 1.0 eq) in methanol (4 mL) was added Pd/C (10 mg, 10% palladium on carbon) and the mixture was stirred at 25° C. for 2 h under H₂ (1100 mmHg). The suspension was filtered through a pad of Celite and the pad was washed with MeOH (3 mL×2), the combined filtrates were concentrated in vacuum to afford the title compound (136.0 mg, quant) as a yellow oil. LCMS: 249.2 [M+H]+, ESI, pos.

### Step E: 1-[(3R)-3-Amino-1-piperidyl]propan-2-ol

A solution of (3R)-1-(2-(benzyloxy)propyl)piperidin-3-amine (136.0 mg, 0.55 mmol, 1.0 eq), Pd/C (10 mg, 10% palladium on carbon) and Pd(OH)2/C (10 mg, 10% palladium on carbon) in methanol (3 mL) was stirred at 70° C. for 16 h under 4500 mmHg. The suspension was filtered through a pad of Celite and the pad was washed with MeOH (5 mL×2). The combined filtrates were concentrated in vacuum to afford the title compound (86 mg, 84% yield) as a yellow oil. 1H NMR (400 MHz, DMSO-d6) δ[ppm]: 3.74-3.68 (m, 1H), 2.77-2.67 (m, 2H), 2.58-2.55 (m, 1H), 2.21-2.09 (m, 2H), 2.03-1.97 (m, 1H), 1.90-1.85 (m, 1H), 1.69-1.66 (m, 1H), 1.64-1.58 (m, 1H), 1.45-1.33 (m, 1H), 1.12-1.03 (m, 1H), 1.01 (d, 3H).

### Step F: 1-((R)-3-((6-Chloro-5-methyl-1,2,4-triazin-3-yl)amino)piperidin-1-yl)propan-2-ol

To a solution of 1-[(3R)-3-amino-1-piperidyl]propan-2-ol (86.0 mg, 0.54 mmol, 1.0 eq), 3,6-dichloro-5-methyl-1,2,4-triazine (CAS #132434-82-3, 89.13 mg, 0.54 mmol, 1.0 eq) in 1,4-dioxane (2 mL) was added DIEA (0.16 mL, 0.98 mmol, 1.8 eq). The reaction mixture was stirred at 25° C. for 16 hours. Upon reaction completion, the reaction mixture was filtered and concentrated under vacuum. The residue was purified by C18 column chromatography (0.1% NH₃·H₂O in water/MeCN, MeCN:54%-46%) and the eluent was lyophilized to afford the title compound (27.0 mg, 17% yield) as a yellow oil. LCMS: m/z 286.1 [M+H]⁺, ESI pos.

### Step G: 5-[5-Methyl-3-[[(3R)-1-(2-hydroxypropyl)-3-piperidyl]amino]-1,2,4-triazin-6-yl]-2,3-dihydrobenzofuran-4-ol

To a solution of 1-((R)-3-((6-chloro-5-methyl-1,2,4-triazin-3-yl)amino)piperidin-1-yl)propan-2-ol (27.0 mg, 0.09 mmol, 1.0 eq), 5-(4,4,5,5-tetramethyl-1,3,2-dioxaborolan-2-yl)-2,3-dihydrobenzofuran-4-ol (Example 1, Step C) (37.15 mg, 0.14 mmol, 1.5 eq), CsF (10.98 mg, 0.19 mmol, 2.0 eq) in 1,4-dioxane (1 mL)/water (0.2 mL) was added XPhos Pd G3 (16.01 mg, 0.02 mmol, 0.2 eq) at 25° C., then stirred at 95° C. for 2 h under N₂. Upon reaction completion, the reaction mixture was cooled to r.t., the combined filtrates were concentrated in vacuum. The residue was purified by preparative HPLC (neutral) to afford the title compound (8.31 mg, 21% yield) as a yellow solid. ¹H NMR (400 MHz, CD₃OD) δ [ppm]: 7.02 (d, 1H), 6.40 (d, 1H), 4.62 (t, 2H), 4.24-4.16 (m, 1H), 3.99-3.91 (m, 1H), 3.20 (t, 2H), 3.08-2.95 (m, 1H), 2.84-2.76 (m, 1H), 2.74-2.50 (m, 1H), 2.46-2.35 (m, 3H), 2.32 (s, 3H), 1.92-1.84 (m, 2H), 1.81-1.67 (m, 1H), 1.64-1.57 (m, 1H), 1.16 (dd, 3H).

### Example 3

### 5-[3-[[(3R)-1-(3-Hydroxypropyl)-3-piperidyl]amino]-5-methyl-1,2,4-triazin-6-yl]-2,3-dihydrobenzofuran-4-ol

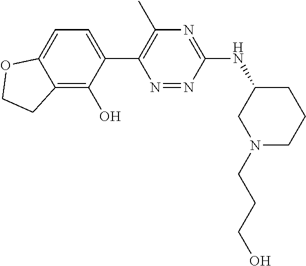 [SMILES: Cc1nc(N[C@@H]2CCCN(CCCO)C2)nnc1-c1ccc2c(c1O)CCO2]

### Step A: Benzyl N-[(3R)-1-(3-benzyloxypropyl)-3-piperidyl]carbamate

To a solution of benzyl N-[(3R)-3-piperidyl]carbamate (CAS: 478646-32-1; 0.5 g, 2.13 mmol, 1.0 eq), CH₃COOH (12.8 mg, 0.21 mmol, 0.1 eq) in DCE (5 mL) was added 3-benzyloxypropanal (CAS: 19790-60-4; 420.5 mg, 2.56 mmol, 1.2 eq) and stirred for 5 mins at 20° C., then NaBH(OAc)₃ (904.6 mg, 4.27 mmol, 5.0 eq) was added and stirred at 20° C. for 1.5 h. The mixture was quenched by ice water (10 ml), and the aqueous layer was extracted with EtOAc (50 mL) twice. The combined organic layers were washed with water (50 mL) and dried over Na₂SO₄, filtered and the filtrate was concentrated under reduced pressure The crude product was purified by column chromatography on silica gel eluting (ethyl acetate/methanol=40:1 to 5:1) to give the title compound (700 mg, 84% yield) as colorless oil. LCMS: m/z 383.1 [M+H]⁺, ESI pos.

### Step B: (3R)-1-(3-Benzyloxypropyl)piperidin-3-amine

To a solution of benzyl N-[(3R)-1-(3-benzyloxypropyl)-3-piperidyl]carbamate (700.0 mg, 1.83 mmol, 1.0 eq) in methanol (2 mL) was added Pd/C (10 mg, 10% palladium on carbon) and Pd(OH)₂/C (10 mg, 10% palladium on carbon) under N₂. After three substitutions using H₂, stirring was continued for 2 hours at 40° C. under H₂ at 1100 mmHg. The mixture was filtered and the filtrate was concentrated under reduced pressure to the title compound (450 mg, 99% yield) as colorless oil. LCMS: m/z 249.0 [M+H]⁺, ESI pos.

### Step C: 3-[(3R)-3-Amino-1-piperidyl]propan-1-ol (CAS: 1704948-85-5)

To a solution of (3R)-1-(3-benzyloxypropyl)piperidin-3-amine (450.0 mg, 2.01 mmol, 1.0 eq) in methanol (10 mL) was added Pd/C (50 mg, 10% palladium on carbon) and under N₂. After three substitutions by H₂, stirring was continued for 2 h at 70° C. under H₂ at 4500 mmHg. The mixture was filtered and the filtrate was concentrated under reduced pressure to afford the title compound (318 mg, 92% yield) as yellow oil.

### Step D: 3-[(3R)-3-[(6-Chloro-5-methyl-1,2,4-triazin-3-yl)amino]-1-piperidyl]propan-1-ol

To a solution of 3-[(3R)-3-amino-1-piperidyl]propan-1-ol (57.9 mg, 0.37 mmol, 1.2 eq), DIEA (0.08 mL, 0.46 mmol, 1.5 eq) in 1,4-dioxane (1 mL) was added 3,6-dichloro-5-methyl-1,2,4-triazine (50.0 mg, 0.3 mmol, 1.0 eq). The reaction mixture was stirred at 20° C. for 16 hours. The above reaction mixture was concentrated under reduced pressure to get the crude product which was purified by reversed phase flash (CombiFlash 0.1% NH₃·H₂O aqueous-ACN condition) and followed by lyophilization to give the title compound (20.0 mg, 22% yield) as a yellow solid. LCMS: m/z 286.2 [M+H]⁺, ESI pos.

### Step E: 5-[3-[[(3R)-1-(3-Hydroxypropyl)-3-piperidyl]amino]-5-methyl-1,2,4-triazin-6-yl]-2,3-dihydrobenzofuran-4-ol

To a mixture of 5-(4,4,5,5-tetramethyl-1,3,2-dioxaborolan-2-yl)-2,3-dihydrobenzofuran-4-ol (Example 1, Step C) (13.8 mg, 0.05 mmol, 1.5 eq) and 3-[(3R)-3-[(6-chloro-5-methyl-1,2,4-triazin-3-yl)amino]-1-piperidyl]propan-1-ol (10.0 mg, 0.03 mmol, 1.0 eq) in 1,4-dioxane (0.5 mL)/water (0.1 mL), CsF (4.07 mg, 0.07 mmol, 2.0 eq) was added, followed by XPhos Pd G₃ (1.48 mg, 0.0 mmol, 0.05 eq) under N₂ at 25° C. The mixture was stirred at 95° C. for 2 hours. The reaction mixture was cooled to room temperature and concentrated under reduced pressure. The crude was purified by reversed phase flash (CombiFlash 0.1% NH₃HCO₃ water-ACN condition) to afford the title compound (9.1 mg, 66% yield) as a yellow solid. LCMS: m/z 386.3 [M+H]⁺, ESI pos.

### Example 4

### 5-[3-[[(3R)-1-[(1-Hydroxycyclopropyl)methyl]-3-piperidyl]amino]-5-methyl-1,2,4-triazin-6-yl]-2,3-dihydrobenzofuran-4-ol

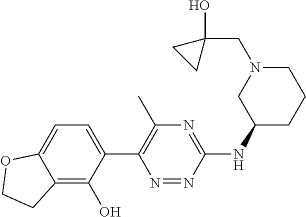 [SMILES: Cc1nc(N[C@@H]2CCCN(CC3(O)CC3)C2)nnc1-c1ccc2c(c1O)CCO2]

### Step A: Methyl 1-((2-(trimethylsilyl)ethoxy)methoxy)cyclopropane-1-carboxylate

To a solution of methyl 1-hydroxycyclopropane-1-carboxylate (5.00 g, 43.06 mmol, 1.0 eq; CAS: 33689-29-1) in DMF (50 mL) was added NaH (2.58 g, 60% 60% w/w in mineral oil, 64.59 mmol, 1.5 eq), then stirred at 0° C. for 20 mins. 2-(Trimethylsilyl)ethoxymethyl chloride (8.38 mL, 47.36 mmol, 1.1 eq) was added slowly and stirred at 25° C. for 1 hour. The above reaction mixture was diluted with NH₄Cl (500 mL), extracted with ethyl acetate (3×200 mL). The combined organic phase was washed with brine (200 mL), dried over anhydrous sodium sulfate, filtered and the filtrate was concentrated under reduced pressure. The crude product was then purified by column chromatography on silica gel (petroleum ether/ethyl acetate: 1/0 to 10/1) to afford the title compound (2.10 g, 20% yield) as yellow oil. ¹H NMR (400 MHz, CD₃OD) δ [ppm]: 4.79 (s, 2H), 3.71 (s, 3H), 3.67 (t, 2H), 1.30-1.25 (m, 4H), 0.90 (t, 2H), 0.03 (s, 9H).

### Step B: (1-((2-(Trimethylsilyl)ethoxy)methoxy)cyclopropyl)methanol

To a solution of methyl 1-((2-(trimethylsilyl)ethoxy)methoxy)cyclopropane-1-carboxylate (1.10 g, 4.46 mmol, 1.0 eq) in DCM (20 mL) was added dropwise DIBAl-H (13.39 mL, 13.39 mmol, 3.0 eq) at −78° C. under N₂ and the mixture was stirred at −78° C. for 1 h. The above reaction mixture was diluted with saturated NH₄Cl (100 mL) at 0° C. and DCM (20 mL), filtered and the filtrate was extracted with DCM (20 mL×3). The combined organic phase was washed with brine (50 mL), dried over anhydrous sodium sulfate, filtered and the filtrate was concentrated under reduced pressure. The crude product was purified by column chromatography on silica gel (petroleum ether/ethyl acetate: 1/0 to 10/1) to afford the title compound (0.55 g, 2.5 mmol, 56% yield) as yellow oil. ¹H NMR (400 MHz, DMSO-d₆) δ [ppm]: 4.68 (s, 2H), 4.60 (t, 1H), 3.57-3.49 (m, 4H), 0.85 (t, 1H), 0.75-0.68 (m, 2H), 0.58-0.52 (m, 2H), 0 (s, 9H).

### Step C: 1-((2-(Trimethylsilyl)ethoxy)methoxy)cyclopropane-1-carbaldehyde

Oxalyl chloride (0.28 mL, 3.30 mmol, 2.0 eq) was added into a solution of DMSO (0.23 mL, 3.3 mmol, 2.0 eq) in DCM (7 mL) at −70° C. and stirred for 10 mins at −70° C. Then the solution of (1-((2-(trimethylsilyl)ethoxy)methoxy)cyclopropyl)methanol (0.360 g, 1.65 mmol, 1.0 eq) in DCM (2 mL) was added dropwise into the above solution and stirred at −70° C. for 0.5 hour. TEA (1.8 mL, 12.93 mmol, 7.84 eq) was added and stirring was continued at 0° C. for 30 mins. The above reaction mixture was warmed to r.t., diluted with water (50 mL), extracted with DCM (30 mL×3). The combined organic phase was washed with brine (50 mL), dried over anhydrous sodium sulfate, filtered and the filtrate was concentrated under reduced pressure. The crude product was purified by column chromatography on silica gel (petroleum ether/ethyl acetate: 1/0 to 10/1) to afford the title compound (0.18 g, 50% yield) as yellow oil. ¹H NMR (400 MHz, DMSO-d₆) δ [ppm]: 9.26 (s, 1H), 4.71 (s, 2H), 3.66 (t, 2H), 1.34-1.32 (m, 2H), 1.30-1.28 (m, 2H), 0.86 (t, 2H), 0.01 (s, 9H).

### Step E: tert-Butyl (R)-3-((6-(4-hydroxy-2,3-dihydrobenzofuran-5-yl-5-methyl-1,2,4-triazin-3-yl)amino)piperidine-1-carboxylate

To a solution of tert-butyl (3R)-3-[(6-chloro-5-methyl-1,2,4-triazin-3-yl)amino]piperidine-1-carboxylate (700 mg, 2.14 mmol, 1.0 eq; preparation as described in Example 1, Step D), 5-(4,4,5,5-tetramethyl-1,3,2-dioxaborolan-2-yl)-2,3-dihydrobenzofuran-4-ol (Example 1, Step C) (839 mg, 3.2 mmol, 1.5 eq) and CsF (1.30 g, 8.54 mmol, 4.0 eq) in 1,4-dioxane (20 mL) and water (4 mL) was added XPhos Pd G3 (0.362 g, 0.43 mmol, 0.2 eq) and stirring was continued at 95° C. for 3 hours under N₂ atmosphere. The above reaction mixture was cooled to r.t., diluted with ethyl acetate (100 mL), filtered and the filtrate was concentrated under reduced pressure. The crude product was purified by column chromatography on silica gel (petroleum ether/ethyl acetate: 1/0 to 1/1) to afford the title compound (0.750 g, 82% yield) as yellow solid. ¹H NMR (400 MHz, DMSO-d₆) δ [ppm]: 9.38 (s, 1H), 7.53 (s, 1H), 6.95 (d, 1H), 6.38 (d, 1H), 4.57 (t, 2H), 3.88-3.82 (m, 2H), 3.65-3.30 (m, 1H), 3.15 (t, 2H), 3.10-2.74 (m, 1H), 2.20 (s, 3H), 1.99-1.93 (m, 1H), 1.81-1.71 (m, 1H), 1.63-1.50 (m, 1H), 1.47-1.42 (m, 1H), 1.34 (s, 9H).

### Step F: (R)-5-(5-Methyl-3-(piperidin-3-ylamino)-1,2,4-triazin-6-yl)-2,3-dihydrobenzofuran-4-ol

A solution of tert-butyl (R)-3-((6-(4-hydroxy-2,3-dihydrobenzofuran-5-yl)-5-methyl-1,2,4-triazin-3-yl)amino)piperidine-1-carboxylate (0.450 g, 1.05 mmol, 1.0 eq) in DCM (3 mL)/TFA (3.0 mL) was stirred at 25° C. for 1 h. The solvent was removed by nitrogen flow to get the crude product, then the pH was adjusted to about 7, with NH₃·H₂O solution. The crude product was purified by reversed phase flash (0.1% NH₃·H₂O, water-MeCN) to afford the title compound (0.346 g, 77% yield) as yellow solid. LCMS: m/z 328.2 [M+H]⁺, ESI pos.

### Step G: (R)-5-(5-Methyl-3-((1-((1-((2-(trimethylsilyl) ethoxy) methoxy) cyclopropyl) methyl) piperidin-3-yl)amino)-1,2,4-triazin-6-yl)-2,3-dihydrobenzofuran-4-ol

A mixture of 1-((2-(trimethylsilyl)ethoxy)methoxy)cyclopropane-1-carbaldehyde (0.171 g, 0.79 mmol, 1.3 eq), AcOH (3.7 mg, 0.06 mmol, 0.1 eq), (R)-5-(5-methyl-3-(piperidin-3-ylamino)-1,2,4-triazin-6-yl)-2,3-dihydrobenzofuran-4-ol (200 mg, 0.61 mmol, 1.0 eq) in DCE (2 mL) was stirred for 10 minutes at 25° C., then NaBH₃CN (7.67 mg, 1.22 mmol, 2.0 eq) was added and stirred at 40° C. for 1 h. The solvent was removed by nitrogen flow. The crude product was purified by reversed phase flash (0.1% NH₃·H₂O, water-ACN), to afford the title compound (120 mg, 0.23 mmol, 37% yield) as yellow solid. LCMS: m/z 528.4 [M+H]⁺, ESI pos.

### Step H: 5-[3-[[(3R)-1-[(1-Hydroxycyclopropyl)methyl]-3-piperidyl]amino]-5-methyl-1,2,4-triazin-6-yl]-2,3-dihydrobenzofuran-4-ol

A solution of (R)-5-(5-methyl-3-((1-((1-((2-(trimethylsilyl)ethoxy) methoxy) cyclopropyl) methyl)piperidin-3-yl)amino)-1,2,4-triazin-6-yl)-2,3-dihydrobenzofuran-4-ol (40 mg, 0.08 mmol, 1.0 eq) in DCM (1 mL)/TFA (1.0 mL) was stirred at 25° C. for 1 hour. The solvent was removed by nitrogen flow. The pH was then adjusted to 7 with NH₃·H₂O and the crude was purified by reversed phase flash (0.1% NH₃·H₂O water-ACN) to yield the title compound (9.89 mg, 0.02 mmol, 31% yield) as yellow solid. LCMS: m/z 398.3 [M+H]⁺, ESI pos.

### Example 5

### 5-[3-[[(3R)-1-(2-Methoxyethyl)-3-piperidyl]amino]-5-methyl-1,2,4-triazin-6-yl]-2,3-dihydrobenzofuran-4-ol; formic acid salt

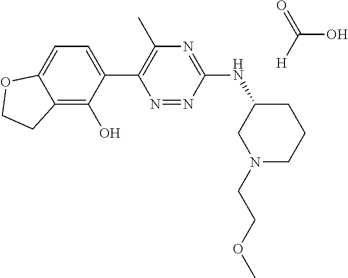

### Step A: Benzyl N-[(3R)-1-(2-methoxyethyl)-3-piperidyl]carbamate

To a solution of benzyl N-[(3R)-3-piperidyl]carbamate (CAS #478646-32-1, 0.5 g, 2.13 mmol, 1.0 eq), N-ethyl-N-isopropylpropan-2-amine (0.75 mL, 4.27 mmol, 2.0 eq) in acetonitrile (5 mL) was added 2-bromoethyl methyl ether (CAS: 6482-24-2; 0.3 mL, 3.2 mmol, 1.5 eq) and the reaction mixture was stirred at 50° C. for 6 hours. Upon reaction completion, the reaction mixture was cooled to r.t. Ethyl acetate (5 mL) and water (5 mL) were added and layers were separated. The aqueous phase was extracted with ethyl acetate (5 mL×3). Combined extracts were washed with brine (5 mL×3), dried over Na₂SO₄, filtered, and concentrated under vacuum. The residue was purified by column chromatography on silica gel (petroleum ether/ethyl acetate: 1/1 to 1/2) to afford the title compound (520.0 mg, 83% yield) as orange solid. LCMS: m/z 293.1 [M+H]⁺, ESI pos.

### Step B: (3R)-1-(2-Methoxyethyl) piperidin-3-amine

To a solution of benzyl N-[(3R)-1-(2-methoxyethyl)-3-piperidyl]carbamate (470.0 mg, 1.61 mmol, 1.0 eq) in methanol (5 mL) was added Pd/C (50.0 mg, 10% palladium on carbon) and Pd(OH)₂/C (52.22 mg, 10% palladium on carbon) under N₂. After three substitutions by H₂, the mixture was then stirred for 2 hours at 20° C. under H₂ at 1100 mmHg. Upon reaction completion, the reaction mixture was filtered, and the mother liquor was concentrated under vacuum to afford the title compound (240.0 mg, 94% yield) as yellow oil. ¹H-NMR (400 MHz, DMSO-d₆) δ [ppm]: 3.39 (t, 2H), 3.22 (s, 3H), 2.77-2.71 (m, 1H), 2.67-2.61 (m, 1H), 2.60-2.54 (m, 1H), 2.44-2.39 (m, 2H), 1.91-1.84 (m, 1H), 1.72-1.67 (m, 1H), 1.67-1.62 (m, 1H), 1.59-1.52 (m, 1H), 1.40 (s, 1H), 0.95-0.85 (m, 1H).

### Step C: 6-Chloro-N-[(3R)-1-(2-methoxyethyl)-3-piperidyl]-5-methyl-1,2,4-triazin-3-amine

To a solution of (3R)-1-(2-methoxyethyl) piperidin-3-amine (231.6 mg, 1.46 mmol, 1.2 eq), N-ethyl-N-isopropylpropan-2-amine (0.38 mL, 2.2 mmol, 1.8 eq) in 1,4-dioxane (3 mL) was added 3,6-dichloro-5-methyl-1,2,4-triazine (CAS #132434-82-3, 200.0 mg, 1.22 mmol, 1.0 eq), The reaction mixture was stirred at 20° C. for 16 h. Upon the reaction completion, the mixture was filtered and concentrated under vacuum. The residue was purified by C18 column chromatography (0.1% TFA in water/acetonitrile, acetonitrile: 30%-40%). The crude product was purified by column chromatography on silica gel (petroleum ether/ethyl acetate: 1/0 to 10/1) and then purified by preparative TLC (dichloromethane/methanol: 5/1) to afford the title compound (30.0 mg, 9% yield) as colorless oil. LCMS: m/z 286.2 [M+H]+, ESI pos.

### Step D: 5-[3-[[(3R)-1-(2-Methoxyethyl)-3-piperidyl]amino]-5-methyl-1,2,4-triazin-6-yl]-2,3-dihydrobenzofuran-4-ol; formic acid salt

To a solution of 6-chloro-N-[(3R)-1-(2-methoxyethyl)-3-piperidyl]-5-methyl-1,2,4-triazin-3-amine (30.0 mg, 0.1 mmol, 1.0 eq), 5-(4,4,5,5-tetramethyl-1,3,2-dioxaborolan-2-yl)-2,3-dihydrobenzofuran-4-ol (Example 1, Step C) (41.3 mg, 0.16 mmol, 1.5 eq), CsF (63.8 mg, 0.42 mmol, 4.0 eq) in 1,4-dioxane (1 mL) and water (0.2 mL) was added XPhos Pd G3 (17.8 mg, 0.02 mmol, 0.2 eq) at 20° C. under N₂. The reaction mixture was stirred at 80° C. for 2 hours. Upon reaction completion, the mixture was cooled to r.t., filtered and concentrated under vacuum. The residue was purified by preparative HPLC (column: Phenomenex luna C₁₈ 150*25 mm*10 um; condition:water (formic acid)-acetonitrile; Begin B: 7; End B: 37; Gradient Time (min): 10; 100% B Hold Time (min): 2; Flow Rate (mL/min): 25) to afford the title compound (18.2 mg, 40% yield) as brown gum. LCMS: m/z 386.1 [M+H]⁺, ESI pos.

### Example 6

### (3S,5R)-1-Ethyl-5-[[6-(4-hydroxy-2,3-dihydrobenzofuran-5-yl)-5-methyl-1,2,4-triazin-3-yl]amino]piperidin-3-ol

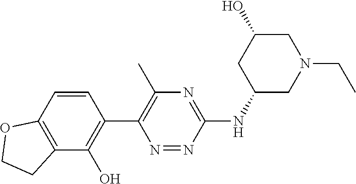 [SMILES: CCN1C[C@@H](O)C[C@@H](Nc2nnc(-c3ccc4c(c3O)CCO4)c(C)n2)C1]

### Step A: tert-Butyl (3R,5S)-3-[(6-chloro-5-methyl-1,2,4-triazin-3-yl)amino]-5-hydroxy-piperidine-1-carboxylate

To a solution of 3,6-dichloro-5-methyl-1,2,4-triazine (CAS #132434-82-3, 243 mg, 1.48 mmol, 1.0 eq) in 1,4-dioxane (5 mL) was added at ambient temperature (3R,5S)-3-amino-5-hydroxy-piperidine-1-carboxylic acid tert-butyl ester (CAS #1932513-59-1, 396 mg, 1.78 mmol, 1.2 eq) and N,N-diisopropylethylamine (345 mg, 0.47 mL, 2.67 mmol, 1.8 eq) resulting in a dark brown solution. The reaction mixture was stirred at 23° C. for 60 hours. Afterwards, the reaction mixture was quenched with half-saturated aq. NH₄Cl-solution (50 mL) and extracted with ethyl acetate (2×50 mL). The organic layers were washed with water (80 mL) and brine (80 mL). The combined organic extracts were dried over sodium sulfate, filtered and the solvent was evaporated under reduced pressure. The residue was purified by flash chromatography (silica gel, 25 g, 0-80% ethyl acetate in heptane) to afford the title compound (345 mg, 68% yield) as a light yellow oil. LCMS: m/z 344.2 ([{35Cl}M+H]⁺), 346.2 ([{37Cl}M+H]⁺), ESI pos.

### Step B: (3S,5R)-5-[(6-Chloro-5-methyl-1,2,4-triazin-3-yl)amino]piperidin-3-ol; hydrochloride

To a solution of tert-butyl (3R,5S)-3-[(6-chloro-5-methyl-1,2,4-triazin-3-yl)amino]-5-hydroxy-piperidine-1-carboxylate (Example 6, step A) (345 mg, 1.0 mmol, 1.0 eq) in dichloromethane (10 mL) and methanol (5 mL) was added at room temperature 4 M HCl in 1,4-dioxane (3.01 g, 2.51 mL, 10.03 mmol, 10.0 eq) dropwise. The reaction mixture was stirred at 23° C. for 16 h. The reaction mixture was concentrated in vacuo to afford the title compound (312 mg, 100% yield) as a light brown foam, which was used without further purification. LCMS: m/z 244.1 ([{35Cl}M+H]+), 246.1 ([{37Cl}M+H]+), ESI pos.

### Step C: (3S,5R)-5-[(6-Chloro-5-methyl-1,2,4-triazin-3-yl)amino]-1-ethyl-piperidin-3-ol

To a suspension of (3S,5R)-5-[(6-chloro-5-methyl-1,2,4-triazin-3-yl)amino]piperidin-3-ol; hydrochloride (Example 6, step B) (312 mg, 1.0 mmol, 1.0 eq)) in dichloromethane (15 mL) was added at 0° C. acetaldehyde (CAS #75-07-0); 110 mg, 0.14 mL, 2.51 mmol, 2.5 eq)) followed by the addition of sodium acetate (CAS #127-09-3, 206 mg, 2.51 mmol, 2.5 eq) and sodium triacetoxyborohydride (CAS #56553-60-7; 382 mg, 1.8 mmol, 1.8 eq). The reaction mixture was stirred at 0° C. for 5 minutes and at 23° C. for 3 hours. The reaction mixture was carefully basified with saturated NaHCO₃-solution (25 mL), then extracted with dichloromethane (3×60 mL). The combined organic layers were dried over sodium sulfate, filtered and concentrated in vacuo. The crude product was adsorbed on ISOLUTE HM-N and purified by flash chromatography (silica gel, 25 g, gradient 0-100% (dichloromethane:methanol:NH₄OH 110:10:1) in dichloromethane) to afford the title compound (137 mg, 50% yield) as a light brown foam. LCMS: m/z 272.1 ([{35Cl}M+H]⁺), 274.1 ([{37Cl}M+H]⁺), ESI pos.

### Step D: (3S,5R)-1-Ethyl-5-[[6-(4-hydroxy-2,3-dihydrobenzofuran-5-yl)-5-methyl-1,2,4-triazin-3-yl]amino]piperidin-3-ol

A mixture of (3S,5R)-5-[(6-chloro-5-methyl-1,2,4-triazin-3-yl)amino]-1-ethyl-piperidin-3-ol (Example 6, step C) (84 mg, 0.31 mmol, 1.0 eq) and 5-(4,4,5,5-tetramethyl-1,3,2-dioxaborolan-2-yl)-2,3-dihydrobenzofuran-4-ol (Example 1, step C) (154 mg, 0.56 mmol, 1.8 eq) and potassium carbonate (205 mg, 1.48 mmol, 4.8 eq) was dissolved in 1,4-dioxane (2 mL) and water (1 mL). The sealable tube was flushed with argon and 1,1′-bis(diphenylphosphino)ferrocene-palladium(ii)dichloride dichloromethane complex (CAS #95464-05-4, 38 mg, 0.046 mmol, 0.15 eq) was added. After flushing again with argon, the sealed tube was stirred at 90° C. for 16 hours. The reaction mixture was cooled to room temperature and quenched with water (50 mL) and saturated NH₄Cl-solution (50 mL), then extracted with dichloromethane (3×50 mL). The organic layers were washed with brine (50 mL), dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 110:10:1) in dichloromethane) followed by preparative HPLC (column: Gemini NX, 12 nm, 5 μm, 100×30 mm; MeCN/water (+0.1% TEA)) to afford the title compound (43 mg, 37% yield) as a yellow amorphous freeze-dried solid. LCMS: m/z 372.2 [M+H]⁺, ESI pos.

### Example 7

### 5-[5-Methyl-3-[[(3R)-1-(2-methoxypropyl)-3-piperidyl]amino]-1,2,4-triazin-6-yl]-2,3-dihydrobenzofuran-4-ol

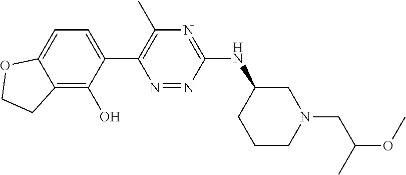 [SMILES: COC(C)CN1CCC[C@@H](Nc2nnc(-c3ccc4c(c3O)CCO4)c(C)n2)C1]

### Step A: 2-Methoxypropyl ethanesulfonate

To a solution of 2-methoxypropanol (1.10 g, 12.2 mmol, 1.0 eq), DIEA (4.72 g, 36.6 mmol, 3.0 eq) in DCM (10 mL) was added ethane sulfonyl chloride (1.73 mL, 18.3 mmol) at 0° C., then stirred at 20° C. for 1 h. Upon reaction completion, the above reaction mixture was diluted with water (30 mL), extracted with ethyl acetate (50 mL×3). The combined organic phase was washed with brine (20 mL), dried over anhydrous sodium sulfate, filtered and the filtrate was concentrated under reduced pressure. The residue was purified by column chromatography on silica gel (petroleum ether/ethyl acetate: 1/0 to 1/1) to afford the title compound (2.3 g, 98% yield) as a black solid. ¹H NMR (400 MHz, CD₃OD) δ [ppm]: 4.23-4.19 (m, 1H), 4.15-4.11 (m, 1H), 3.67-3.59 (m, 1H), 3.41 (s, 3H), 3.20 (q, 2H), 1.45 (t, 3H), 1.21 (d, 3H).

### Step B: Benzyl N-[(3R)-1-(2-methoxypropyl)-3-piperidyl]carbamate

To a solution of benzyl N-[(3R)-3-piperidyl]carbamate (CAS #478646-32-1, 500.0 mg, 2.13 mmol, 1.0 eq), TEA (0.5 g, 4.91 mmol, 2.3 eq) in ACN (5 mL) was added 2-methoxypropyl ethanesulfonate (532.2 mg, 2.77 mmol, 1.3 eq) at 0° C., then stirred at 80° C. for 16 h. The above reaction mixture was concentrated in vacuum. The residue was purified by column chromatography on silica gel (petroleum ether/ethyl acetate: 1/0 to 1/1) to afford the title compound (190.0 mg, 29% yield) as a brown oil. ¹H NMR (400 MHz, CD₃OD) δ [ppm]: 7.34-7.28 (m, 5H), 5.06 (s, 2H), 3.67-3.61 (m, 1H), 3.55-3.51 (m, 1H), 3.34-3.31 (m, 3H), 2.90-2.82 (m, 1H), 2.70-2.59 (m, 1H), 2.48-2.43 (m, 1H), 2.31-2.15 (m, 1.5H), 2.15-2.05 (m, 1.5H), 1.82-1.68 (m, 2H), 1.63-1.57 (m, 1H), 1.33-1.25 (m, 1H), 1.11 (d, 3H).

### Step C: (3R)-1-(2-Methoxypropyl)piperidin-3-amine

A solution benzyl N-[(3R)-1-(2-methoxypropyl)-3-piperidyl]carbamate (190.0 mg, 0.62 mmol, 1.0 eq), Pd(OH)₂ (10 mg, 10% palladium on carbon) and Pd/C (10 mg, 10% palladium on carbon) in methanol (6 mL) was stirred at 25° C. for 2 h under H₂ at 1100 mmHg. Upon reaction completion, the suspension was filtered through a pad of Celite and the pad was washed with MeOH (5 mL×3). The combined filtrates were concentrated in vacuum to give the desired product as yellow oil, which was used directly without further purification to afford the title compound (75.0 mg, 70% yield) as a yellow oil. ¹H NMR (400 MHz, DMSO-d₆) δ [ppm]: 3.23 (s, 3H), 2.72-2.67 (m, 1H), 2.62-2.55 (m, 2H), 2.36-2.29 (m, 2H), 2.16-2.10 (m, 1H), 1.93-1.87 (m, 1H), 1.71-1.63 (m, 2H), 1.61-1.56 (m, 1H), 1.43-1.34 (m, 2H), 1.04 (d, 3H).

### Step D: 6-Chloro-N-[(3R)-1-(2-methoxypropyl)-3-piperidyl]-5-methyl-1,2,4-triazin-3-amine

To a solution of (3R)-1-(2-methoxypropyl)piperidin-3-amine (75.0 mg, 0.44 mmol, 1.0 eq), DIEA (0.14 mL, 0.78 mmol, 1.8 eq) in 1,4-dioxane (1 mL) was added 3,6-dichloro-5-methyl-1,2,4-triazine (71.4 mg, 0.44 mmol, 1.0 eq) and stirred at 25° C. for 16 h. Upon reaction completion, the reaction mixture quenched with H₂O (10 mL), extracted with EtOAc (20 mL×2), the organic phase was washed with brine (10 mL×2), dried over anhydrous Na₂SO₄, filtered and concentrated in vacuum. The residue was purified by column chromatography (SiO₂, petroleum ether:ethyl acetate, 1:1) to obtain the title compound (36.0 mg, 28% yield) as a dark green oil. LCMS: m/z 300.2 [M+H]⁺, ESI pos.

### Step E: 5-[5-Methyl-3-[[(3R)-1-(2-methoxypropyl)-3-piperidyl]amino]-1,2,4-triazin-6-yl]-2,3-dihydrobenzofuran-4-ol

To a solution of 6-chloro-N-[(3R)-1-(2-methoxypropyl)-3-piperidyl]-5-methyl-1,2,4-triazin-3-amine (18.0 mg, 0.06 mmol, 1.0 eq), 5-(4,4,5,5-tetramethyl-1,3,2-dioxaborolan-2-yl)-2,3-dihydrobenzofuran-4-ol (23.61 mg, 0.09 mmol, 1.5 eq; Example 1, Step C), CsF (6.98 mg, 0.12 mmol, 2.0 eq) in 1,4-dioxane (1 mL)/water (0.2 mL) was added XPhos Pd G3 (10.17 mg, 0.01 mmol, 0.2 eq) under N₂ at 25° C., then stirred at 95° C. for 2 h. Upon reaction completion, the reaction mixture was cooled to r.t., the combined filtrates were concentrated in vacuum. The residue was purified by preparative HPLC (Instrument: ACSWH-GX-M, column: Waters Xbridge 150*25 mm*5 um Mobile phase: A for H₂O (0.1% NH₄HCO₃) and B for acetonitrile Gradient: B 28%-58% in 10 min linearly Flow rate: 30 mL/min Column temperature: R.T. Wavelength: 220 nm/254 nm) to freeze-drying to afford the title compound (9.58 mg, 36% yield) as a yellow oil. LCMS: m/z 400.2 [M+H]⁺, ESI pos.

### Example 8

### 5-[5-Methyl-3-[[(3R)-1-(oxetan-3-yl)-3-piperidyl]amino]-1,2,4-triazin-6-yl]-2,3-dihydrobenzofuran-4-ol

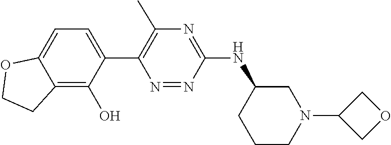 [SMILES: Cc1nc(N[C@@H]2CCCN(C3COC3)C2)nnc1-c1ccc2c(c1O)CCO2]

### Step A: Benzyl (R)-(1-(oxetan-3-yl)piperidin-3-yl)carbamate

To a solution of benzyl (R)-piperidin-3-ylcarbamate (CAS #478646-32-1; 1.0 g, 4.27 mmol, 1.0 eq) in DCE (10 mL) was added oxetan-3-one (CAS #6704-31-0; 0.370 g, 5.12 mmol, 1.2 eq), stirred at 20° C. for 1 hour, then NaBH(OAc)₃ (1.18 g, 5.55 mmol, 1.3 eq) was added and stirred at 20° C. for 1 hour. The above reaction solution was diluted with water (50 mL), extracted with ethyl acetate (20 mL×3). The combined organic phase was washed with brine (50 mL), dried over anhydrous sodium sulfate, filtered and the filtrate was concentrated under reduced pressure. The crude product was purified over column chromatography on silica gel (hexane/EtOAc, 1:0 to 0:1, then EtOAc/MeOH, 1:0 to 5:1) to yield the title compound (1.20 g, 97% yield) as white solid. ¹H NMR (400 MHz, CDCl₃) δ [ppm]: 7.40-7.35 (m, 5H), 5.48-5.26 (m, 1H), 5.11 (s, 2H), 4.67-4.53 (m, 4H), 3.96-3.81 (m, 1H), 3.53-3.39 (m, 1H), 2.47-2.19 (m, 3H), 1.74-1.58 (m, 4H).

### Step B: (R)-1-(Oxetan-3-yl)piperidin-3-amine

To a solution of benzyl (R)-(1-(oxetan-3-yl)piperidin-3-yl)carbamate (200 mg, 0.69 mmol, 1.0 eq) in methanol (4 mL) was added Pd/C (10.0 mg, 10% palladium on carbon) under N₂. After three substitutions by H₂ the mixture was stirred at 20° C. for 1 hour at 1100 mmHg. The mixture was filtered and the filtrate was concentrated under reduced pressure to obtain the title compound (100 mg, 93% yield) as yellow oil. ¹H NMR (400 MHz, CDCl₃) δ [ppm]: 4.67-4.56 (m, 4H), 3.49-3.41 (m, 1H), 2.98-2.87 (m, 1H), 2.66-2.54 (m, 1H), 2.50-2.41 (m, 1H), 2.03 (s, 2H), 1.95-1.81 (m, 2H), 1.79-1.68 (m, 2H), 1.65-1.52 (m, 1H), 1.22-1.08 (m, 1H).

### Step C: (R)-6-Chloro-5-methyl-N-(1-(oxetan-3-yl)piperidin-3-yl)-1,2,4-triazin-3-amine

To a solution of DIPEA (0.157 g, 1.22 mmol, 2.0 eq), (R)-1-(oxetan-3-yl)piperidin-3-amine (0.100 g, 0.64 mmol, 1.05 eq) in 1,4-dioxane (1 mL) was added 3,6-dichloro-5-methyl-1,2,4-triazine (CAS #132434-82-3, 0.100 g, 0.61 mmol, 1.0 eq), then stirred at 20° C. for 12 h. The above reaction solution was diluted with water (50 mL), extracted with ethyl acetate (20 mL×3). The combined organic phase was washed with brine (50 mL), dried over anhydrous sodium sulfate, filtered and the filtrate was concentrated under reduced pressure. The crude was purified by column chromatography on silica gel (hexane/EtOAc, 1:0 to 0:1) to afford the title compound (0.03 g, 0.11 mmol, 17% yield) as yellow solid. ¹H NMR (400 MHz, CD₃OD) δ [ppm]: 4.72-4.57 (m, 4H), 4.14-3.99 (m, 1H), 3.61-3.49 (m, 1H), 2.95-2.81 (m, 1H), 2.68-2.55 (m, 1H), 2.45 (s, 3H), 2.11-2.03 (m, 1H), 2.02-1.92 (m, 2H), 1.87-1.77 (m, 1H), 1.72-1.61 (m, 1H), 1.55-1.41 (m, 1H).

### Step D: 5-[5-Methyl-3-[[(3R)-1-(oxetan-3-yl)-3-piperidyl]amino]-1,2,4-triazin-6-yl]-2,3-dihydrobenzofuran-4-ol

To a solution of (R)-6-chloro-5-methyl-N-(1-(oxetan-3-yl)piperidin-3-yl)-1,2,4-triazin-3-amine (0.015 g, 0.05 mmol, 1.0 eq), 5-(4,4,5,5-tetramethyl-1,3,2-dioxaborolan-2-yl)-2,3-dihydrobenzofuran-4-ol (0.021 g, 0.08 mmol, 1.5 eq; Example 1, Step C), KF (0.015 g, 0.26 mmol, 5.0 eq) in 1,4-dioxane (1 mL) and water (0.2 mL) was added XPhos Pd G3 (9 mg, 0.01 mmol, 0.2 eq) at 25° C. under N₂, then stirred at 90° C. for 4 hours. The above reaction mixture was cooled to r.t., diluted with MeCN (3 mL), filtered and the filtrate was concentrated under reduced pressure. The crude product was purified by prep-HPLC (column: Waters xbridge 150*25 mm 10 um; condition: water (NH₄HCO₃)-ACN Begin B 21 End B 51; gradient time(min) 10 100% B hold time(min) 2; flowrate (mL/min) 25) to yield the title compound (4.35 mg, 20% yield) as a yellow solid. LCMS: m/z 384.1 [M+H]⁺, ESI pos.

### Example 9

### 4-[(3R)-3-[[6-(4-hydroxy-2,3-dihydrobenzofuran-5-yl)-5-methyl-1,2,4-triazin-3-yl]amino]-1-piperidyl]butanoic acid

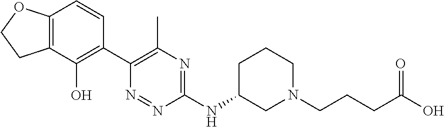 [SMILES: Cc1nc(N[C@@H]2CCCN(CCCC(=O)O)C2)nnc1-c1ccc2c(c1O)CCO2]

### Step A: tert-Butyl (3R)-3-[(6-chloro-5-methyl-1,2,4-triazin-3-yl)amino]piperidine-1-carboxylate

To a solution of 3,6-dichloro-5-methyl-1,2,4-triazine (CAS #132434-82-3, 520 mg, 3.17 mmol, 1.0 eq) in 1,4-dioxane (10 mL) was added at ambient temperature (3R)-3-aminopiperidine-1-carboxylic acid tert-butyl ester (CAS #188111-79-7, 762 mg, 3.81 mmol, 1.2 eq) and N,N-diisopropylethylamine (472 mg, 0.97 mL, 3.66 mmol, 1.80 eq). The reaction mixture was stirred at 23° C. for 48 h. The reaction mixture was quenched with semi saturated NaHCO₃ solution (100 mL) and extracted with ethyl acetate (2×100 mL). The organic layers were washed with water (100 mL) and brine (100 mL). The combined organic extracts were dried over sodium sulfate, filtered off and evaporated. The residue was purified by flash chromatography (silica gel, 12 g, gradient 0% to 50% ethyl acetate in heptane) to afford the title compound (815 mg, 74% yield) as a light yellow gum. LCMS: m/z 326.0 ([{35Cl}M−H]⁻), 328.0 ([{37Cl}M−H]⁻), ESI neg.

### Step B: tert-Butyl (3R)-3-[[6-(4-hydroxy-2,3-dihydrobenzofuran-5-yl)-5-methyl-1,2,4-triazin-3-yl]amino]piperidine-1-carboxylate

In a sealable tube, a mixture of tert-butyl (3R)-3-[(6-chloro-5-methyl-1,2,4-triazin-3-yl)amino]piperidine-1-carboxylate (Example 9, step A) (165 mg, 0.48 mmol, 1.0 eq), 5-(4,4,5,5-tetramethyl-1,3,2-dioxaborolan-2-yl)-2,3-dihydrobenzofuran-4-ol (Example 1, step C) (175 mg, 0.67 mmol, 1.40 eq) and cesium carbonate (467 mg, 1.43 mmol, 3.0 eq) in 1,4-dioxane (4 mL) and water (1 mL) was set under argon, and XPhos Pd G3 gt (61 mg, 0.72 mmol, 0.15 eq) was added finally. The reaction mixture was stirred at 95° C. in the sealed tube for 4 hours. The reaction mixture was cooled to room temperature, then quenched with water (20 mL) and saturated NH₄Cl solution (20 mL) and extracted with ethyl acetate (2×40 mL). The organic layers were washed with brine (40 mL), dried over sodium sulfate, filtered off and concentrated in vacuo. The residue was purified by flash chromatography (silica gel, 12 g, gradient 0% to 90% ethyl acetate in heptane) to afford the title compound (152 mg, 74% yield) as a light yellow foam. LCMS: m/z 428.3 [M+H]⁺, ESI pos.

### Step C: 5-[5-Methyl-3-[[(3R)-3-piperidyl]amino]-1,2,4-triazin-6-yl]-2,3-dihydrobenzofuran-4-ol; dihydrochloride

To a solution of tert-butyl (3R)-3-[[6-(4-hydroxy-2,3-dihydrobenzofuran-5-yl)-5-methyl-1,2,4-triazin-3-yl]amino]piperidine-1-carboxylate (Example 9, step B) (152 mg, 0.36 mmol, 1.0 eq) in CH₂Cl₂ (6 mL) and MeOH (2 mL) was added at +10° C., 4 M HCl in 1,4-dioxane (1.07 g, 0.89 mL, 3.56 mmol, 10 eq) dropwise. The reaction mixture was stirred at 23° C. for 16 h. The reaction mixture was evaporated to dryness to give the title compound (145 mg, 97% yield) as a light yellow foam. LCMS: m/z 326.1 [M−H]⁻, ESI neg.

### Step D: Methyl 4-[(3R)-3-[[6-(4-hydroxy-2,3-dihydrobenzofuran-5-yl)-5-methyl-1,2,4-triazin-3-yl]amino]-1-piperidyl]butanoate

To a suspension of 5-[5-methyl-3-[[(3R)-3-piperidyl]amino]-1,2,4-triazin-6-yl]-2,3-dihydrobenzofuran-4-ol; dihydrochloride (Example 9, step C) (145 mg, 0.34 mmol, 1.0 eq) in dichloromethane (7 mL) was added at 0° C. methyl 4-oxobutanoate (CAS #13865-19-5, 111 mg, 0.10 mL, 0.86 mmol, 2.50 eq) followed by sodium acetate ((CAS #127-09-3, 71 mg, 0.86 mmol, 2.5 eq) and sodium triacetoxyborohyride (CAS #56553-60-7, 131 mg, 0.62 mmol, 1.80 eq). The reaction mixture was stirred at 0° C. for 5 min and at room temperature for 3 hours. To the reaction mixture a saturated NaHCO₃ solution (30 ml) was added and extracted with dichloromethane (3×50 mL). The organic phase was separated and washed with water (20 ml) and brine (20 mL). The combined organic layers were dried over sodium sulfate, filtered off and concentrated in vacuo. The crude product was purified by flash chromatography (silica gel, 12 g, gradient 0% to 50% (dichloromethane:methanol:NH₄OH 110:10:1) in dichloromethane) to afford the title compound (89 mg, 57% yield) as a yellow foam. MS: m/z 426.2 [M−H]⁻, ESI neg.

### Step E: 4-[(3R)-3-[[6-(4-hydroxy-2,3-dihydrobenzofuran-5-yl)-5-methyl-1,2,4-triazin-3-yl]amino]-1-piperidyl]butanoic acid

To solution of methyl 4-[(3R)-3-[[6-(4-hydroxy-2,3-dihydrobenzofuran-5-yl)-5-methyl-1,2,4-triazin-3-yl]amino]-1-piperidyl]butanoate (Example 9, step D) (88 mg, 0.20 mmol, 1.0 eq) in tetrahydrofuran (1 mL) and methanol (0.5 mL) was added at ambient temperature 1 M aqueous LiOH solution (0.59 mL, 0.59 mmol, 3.0 eq) dropwise. The yellow reaction solution was stirred at 23° C. for 16 hours. The reaction mixture was neutralized with 5% citric acid and set to pH=4. The water phase was saturated with solid sodium chloride and extracted with dichloromethane (4×30 mL). The combined organic extracts were dried over sodium sulfate, filtered and the solvent was evaporated in vacuo to afford the title compound (38 mg, 45% yield) as a light yellow solid. MS: m/z 412.1 [M−H]⁻, ESI neg.

### Example 10

### 4-[(3R)-3-[[6-(4-hydroxy-2,3-dihydrobenzofuran-5-yl)-5-methyl-1,2,4-triazin-3-yl]amino]-1-piperidyl]-N-methyl-butanamide

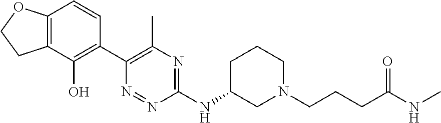 [SMILES: CNC(=O)CCCN1CCC[C@@H](Nc2nnc(-c3ccc4c(c3O)CCO4)c(C)n2)C1]

To a solution of aforementioned 4-[(3R)-3-[[6-(4-hydroxy-2,3-dihydrobenzofuran-5-yl)-5-methyl-1,2,4-triazin-3-yl]amino]-1-piperidyl]butanoic acid (example 9, step E) (6.4 mg, 0.015 mmol, 1.00 eq) and N,N-dimethylformamide (0.4 mL) was added at ambient temperature N,N-diisopropylethylamine (5.7 mg, 0.008 mL, 0.044 mmol, 3.00 eq) followed by HATU (CAS #148893-10-1, 7.30 mg, 0.019 mmol, 1.30 eq). The yellow solution was stirred for 2 minutes, then monomethyl amine hydrochloride (CAS #593-51-1, 1.50 mg, 0.022 mmol, 1.50 eq) was added. The reaction mixture was stirred at 23° C. for 60 h. The reaction mixture was quenched with 0.5 mL of water. The crude product was then purified by preparative HPLC (Column: Gemini NX, 12 nm, 5 μm, 100×30 mm; Condition: MeCN/water+0.1% triethylamine; Gradient: 20% to 80% MeCN in water, runtime 4.5 min) to afford the title compound (3.3 mg, 50% yield) as light yellow solid. LCMS: m/z 427.2 [M+H]⁺, ESI pos.

### Example 11

### 5-[5-Methyl-3-[[(3R)-1-(2-oxazol-2-ylethyl)-3-piperidyl]amino]-1,2,4-triazin-6-yl]-2,3-dihydrobenzofuran-4-ol

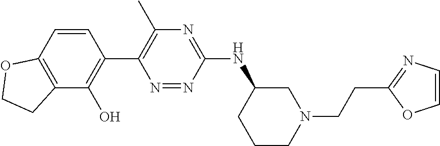 [SMILES: Cc1nc(N[C@@H]2CCCN(CCc3ncco3)C2)nnc1-c1ccc2c(c1O)CCO2]

### Step A: Methyl 3-[(3R)-3-[(6-chloro-5-methyl-1,2,4-triazin-3-yl)amino]-1-piperidyl]propanoate

To a mixture of (6-chloro-5-methyl-1,2,4-triazin-3-yl)-[(3R)-3-piperidyl]amine 1:2 hydrogen chloride (Example 1, step E) (171 mg, 0.455 mmol, 1.0 eq) in tetrahydrofuran, extra dry (2 mL) and N,N-dimethylformamide, extra dry (2 mL) was added N,N-diisopropylethylamine (294 mg, 0.387 mL, 0.0023 mmol, 5.0 eq) followed by dropwise addition of 3-bromopropionic acid methyl ester (CAS #3395-91-3, 114 mg, 0.075 mL, 0.683 mmol, 1.5 eq). The reaction mixture was stirred at 60° C. for 20 hours. The reaction was quenched with water (10 mL) and extracted with ethyl acetate (2×40 mL). The organic layers were washed with brine (20 mL), dried over sodium sulfate, filtered off and concentrated in vacuo. The residue was purified by flash chromatography on silica gel (0% to 50% ethyl acetate in heptane; then ethyl acetate:methanol 9:1 (v/v)) to afford the title compound (108 mg, 72% yield) as a colorless oil. LCMS: m/z 314.1 [M+H]⁺, ESI pos.

### Step B 3-[(3R)-3-[(6-Chloro-5-methyl-1,2,4-triazin-3-yl)amino]-1-piperidyl]-N-(2,2-dimethoxyethyl)propanamide

To a solution of 3-[(3R)-3-[(6-chloro-5-methyl-1,2,4-triazin-3-yl)amino]piperidino]propionic acid methyl ester (Example 11, step A) (108 mg, 0.33 mmol, 1.0 eq) in tetrahydrofuran (2 mL) and methanol (1 mL) was added dropwise 1 M aq. lithium hydroxide (0.425 mL, 0.425 mmol, 1.3 eq). The reaction mixture was stirred at room temperature for 16 hours. Then the reaction mixture was concentrated. The residue was taken up twice in toluene, concentrated and dried at high vacuum at 50° C. for 1 hour. The residue was dissolved in N,N-dimethylformamide, extra dry (3 mL) and at ambient temperature N-ethyldiisopropylamine (CAS #7087-98-5, 211.3 mg, 0.278 mL, 1.6 mmol, 5.0 eq) and HATU (CAS #148893-10-1, 187 mg, 0.49 mmol, 1.5 eq) were added. After stirring for 2 min 2,2-dimethoxyethylamine (62 mg, 0.064 mL, 0.589 mmol, 1.8 eq) was added. The reaction mixture was stirred at 23° C. for 3 h. The reaction mixture was quenched with water (15 mL) and extracted with ethylacetate (2×30 mL). The organic layers were washed with brine (30 mL). The combined organic extracts were dried over sodium sulfate, filtered off and concentrated in vacuo. The crude product was purified by flash chromatography on silica gel (gradient 0% to 50% (dichloromethane:methanol:NH₄OH 110:10:1) in dichloromethane) to afford the title compound (137 mg, 97% yield) as a light brown oil. LCMS: m/z 387.3 [M+H]⁺, ESI pos.

### Step C: 6-Chloro-5-methyl-N-[(3R)-1-(2-oxazol-2-ylethyl)-3-piperidyl]-1,2,4-triazin-3-amine

A mixture of 3-[(3R)-3-[(6-chloro-5-methyl-1,2,4-triazin-3-yl)amino]piperidino]-N-(2,2-dimethoxyethyl)propionamide (Example 11, step B) (137 mg, 0.32 mmol, 1.0 eq) and Eaton's reagent (CAS #39394-84-8, 3.79 g, 2.5 mL, 16 mmol, 50 eq) was stirred at 100° C. overnight. The reaction mixture was cooled to room temperature, added dropwise onto saturated aq. NaHCO₃-solution (100 mL) (strong gas evolution; pH check: ~7) and extracted with dichloromethane (3×60 mL). The combined organic extracts were dried over sodium sulfate, filtered off and concentrated in vacuo. The crude product was purified by flash chromatography on silica gel (gradient 0% to 50% (dichloromethane:methanol:NH₄OH 110:10:1) in dichloromethane) to afford the title compound (34 mg, 31% yield) as colorless oil. LCMS: m/z 323.2 [M+H]⁺, ESI pos.

### Step D: 5-[5-Methyl-3-[[(3R)-1-(2-oxazol-2-ylethyl)-3-piperidyl]amino]-1,2,4-triazin-6-yl]-2,3-dihydrobenzofuran-4-ol

A mixture of (6-chloro-5-methyl-1,2,4-triazin-3-yl)-[(3R)-1-(2-oxazol-2-ylethyl)-3-piperidyl]-amine (Example 11, step C) (34 mg, 0.1 mmol, 1.0 eq), 5-(4,4,5,5-tetramethyl-1,3,2-dioxaborolan-2-yl)coumaran-4-ol (Example 1, step C) (39 mg, 0.15 mmol, 1.5 eq) and cesium carbonate (88 mg, 0.3 mmol, 3.0 eq) in 1,4-dioxane (1.6 mL) and water (0.4 mL) was set under argon, and XPhos Pd g3 gt (12.7 mg, 0.015 mmol, 0.15 eq) was added. The reaction mixture was stirred at 90° C. in a sealed tube for 3 h. The reaction mixture was cooled to room temperature, quenched with water (20 mL) and sat. aq. NH₄Cl (20 mL) and extracted with ethyl acetate (2×40 mL). The organic layers were washed with brine (40 mL), dried over sodium sulfate, filtered off and concentrated in vacuo. The crude product was purified by flash chromatography on silica gel (gradient 0% to 80% (dichloromethane:methanol:NH₄OH 110:10:1) in dichloromethane) to afford the title compound (24 mg, 54% yield) as a light brown foam. LCMS: m/z 423.3 [M+H]⁺, ESI pos.

### Example 12

### 4-[(3R)-3-[[6-(4-Hydroxy-2,3-dihydrobenzofuran-5-yl)-5-methyl-1,2,4-triazin-3-yl]amino]-1-piperidyl]-N,N-dimethyl-butanamide

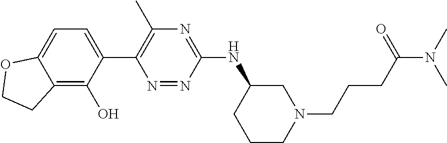 [SMILES: Cc1nc(N[C@@H]2CCCN(CCCC(=O)N(C)C)C2)nnc1-c1ccc2c(c1O)CCO2]

To a solution of 4-[(3R)-3-[[6-(4-hydroxycoumaran-5-yl)-5-methyl-1,2,4-triazin-3-yl]amino]piperidino]butyric acid (Example 9, step E) (43 mg, 0.094 mmol, 1.0 eq) and N,N-dimethylformamide, extra dry (0.3 mL) was added at ambient temperature N,N-diisopropylethylamine (60.5 mg, 0.08 mL, 0.468 mmol, 5.0 eq) followed by HATU (CAS #148893-10-1, 55.0 mg, 0.14 mmol, 1.5 eq). The yellow solution was stirred for 5 min, then dimethylamine hydrochloride (CAS #506-89-2, 13.7 mg, 0.168 mmol, 1.8 eq) was added. The reaction mixture was stirred at 23° C. for 60 hours. The reaction mixture was quenched with water (10 mL) and extracted with ethyl acetate (2×20 mL). The organic layers were washed with brine (20 mL), dried over Na₂SO₄, filtered off and concentrated in vacuo. The crude product was purified by flash chromatography on silica gel (gradient 0% to 50% (dichloromethane:methanol:NH₄OH 110:10:1) in dichloromethane). The product was then purified by preparative HPLC (Column: Gemini NX, 12 nm, 5 μm, 100×30 mm; Condition: ACN/water+0.1% TEA; Gradient: ACN in water, runtime 4.5 min) to afford the title compound. (9 mg, 21% yield) as light yellow freeze-dried solid. LCMS: m/z 441.5 [M+H]⁺, ESI pos.

### Example 13

### 4-[(3R)-3-[[6-(4-Hydroxy-2,3-dihydrobenzofuran-5-yl)-5-methyl-1,2,4-triazin-3-yl]amino]-1-piperidyl]-1-pyrrolidin-1-yl-butan-1-one

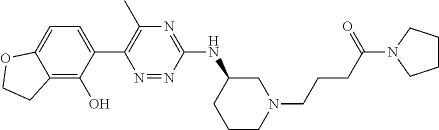 [SMILES: Cc1nc(N[C@@H]2CCCN(CCCC(=O)N3CCCC3)C2)nnc1-c1ccc2c(c1O)CCO2]

To a solution of 4-[(3R)-3-[[6-(4-hydroxycoumaran-5-yl)-5-methyl-1,2,4-triazin-3-yl]amino]piperidino]butyric acid (Example 9, step E) (30.0 mg, 0.065 mmol, 1.0 eq) and N,N-dimethylformamide, extra dry (0.265 mL) was added at ambient temperature N,N-diisopropylethylamine (42.2 mg, 0.056 mL, 0.327 mmol, 5.0 eq) followed by HATU (CAS #148893-10-1, 38.4 mg, 0.098 mmol, 1.5 eq). The yellow solution was stirred for 5 min, then pyrrolidine (CAS #123-75-1, 8.4 mg, 0.0097 mL, 0.118 mmol, 1.8 eq) was added. The reaction mixture was stirred at 23° C. for 16 h. Then, HATU (38.4 mg, 0.098 mmol, 1.5 eq), N,N-diisopropylethylamine (42.2 mg, 0.059 mL, 0.327 mmol, 5.0 eq) and 4-[(3R)-3-[[6-(4-hydroxycoumaran-5-yl)-5-methyl-1,2,4-triazin-3-yl]amino]piperidino]butyric acid (30 mg, 0.065 mmol, 1.0 eq) were added to the reaction mixture and stirring was continued at 23° C. for 48 h. The reaction mixture was quenched with 0.5 mL of water. The crude product was then purified by preparative HPLC (Column: Gemini NX, 12 nm, 5 μm, 100×30 mm; Condition: ACN/water+0.1% HCOOH; Gradient: ACN in water, runtime 4.5 min) to afford the title compound (7.3 mg, 22% yield) as yellow solid. LCMS: m/z 467.4 [M+H]⁺, ESI pos.

### Example 14

### 4-[(3R)-3-[[6-(4-4-[(3R)-3-[[6-(4-Hydroxy-2,3-dihydrobenzofuran-5-yl)-5-methyl-1,2,4-triazin-3-yl]amino]-1-piperidyl]-N-(2-hydroxyethyl)-N-methyl-butanamide

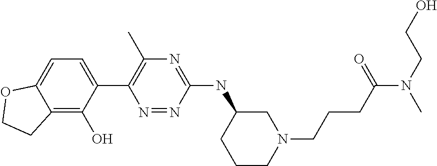 [SMILES: Cc1nc(N[C@@H]2CCCN(CCCC(=O)N(C)CCO)C2)nnc1-c1ccc2c(c1O)CCO2]

To a solution of 4-[(3R)-3-[[6-(4-hydroxycoumaran-5-yl)-5-methyl-1,2,4-triazin-3-yl]amino]piperidino]butyric acid (Example 9, step E) (30 mg, 0.065 mmol, 1.0 eq) and N,N-dimethylformamide, extra dry (0.265 mL) was added at ambient temperature N,N-diisopropylethylamine (42.2 mg, 0.056 mL, 0.327 mmol, 5.0 eq) followed by HATU (CAS #148893-10-1, 38.4 mg, 0.098 mmol, 1.5 eq). The yellow solution was stirred for 2 min, then 2-(methylamino)ethanol (CAS #109-83-1, 8.83 mg, 0.0094 mL, 0.118 mmol, 1.8 eq) was added. The reaction mixture was stirred at 23° C. for 16 h. HATU (38.4 mg, 0.098 mmol, 1.5 eq), N,N-diisopropylethylamine (42.2 mg, 0.0556 mL, 0.327 mmol, 5.0 eq) and 4-[(3R)-3-[[6-(4-hydroxycoumaran-5-yl)-5-methyl-1,2,4-triazin-3-yl]amino]piperidino]butyric acid (example 9, step E) (30 mg, 0.065 mmol, 1.0 eq) were added to the reaction mixture. The reaction mixture was quenched with 0.5 mL of water. The crude product was then purified by preparative HPLC (Column: YMC-Triart C18, 12 nm, 5 μm, 100×30 mm; Condition: ACN/water+0.1% HCOOH; Gradient: ACN in water, runtime 4.5 min) to afford the title compound (6 mg, 18% yield) as yellow solid. LCMS: m/z 471.4 [M+H]⁺, ESI pos.

### Example 15

### 1-(3-Hydroxypyrrolidin-1-yl)-4-[rac-(3R)-3-[[6-(4-hydroxy-2,3-dihydrobenzofuran-5-yl)-5-methyl-1,2,4-triazin-3-yl]amino]-1-piperidyl]butan-1-one; formic acid

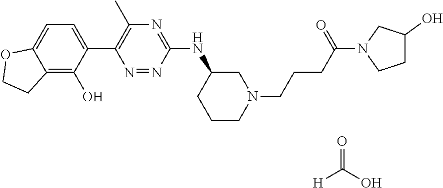 [SMILES: Cc1nc(N[C@@H]2CCCN(CCCC(=O)N3CCC(O)C3)C2)nnc1-c1ccc2c(c1O)CCO2 ; O=CO]

To a solution of 4-[(3R)-3-[[6-(4-hydroxycoumaran-5-yl)-5-methyl-1,2,4-triazin-3-yl]amino]piperidino]butyric acid (Example 9, step E) (34 mg, 0.074 mmol, 1.0 eq) and N,N-dimethylformamide, extra dry (0.3 mL) was added at ambient temperature N,N-diisopropylethylamine (47.8 mg, 0.063 uL, 0.37 mmol, 5.0 eq) followed by HATU (CAS #148893-10-1, 43.5 mg, 0.111 mmol, 1.5 eq). The yellow solution was stirred for 2 min, then DL-pyrrolidin-3-ol (CAS #40499-83-0, 11.6 mg, 0.011 mL, 0.133 mmol, 1.8 eq) was added. The reaction mixture was stirred at 23° C. for 60 h. The reaction mixture was quenched with 0.5 mL of water and concentrated. The product was purified by preparative HPLC (Column: Gemini NX, 12 nm, 5 μm, 100×30 mm; Condition: ACN/water+0.1% HCOOH; Gradient: ACN in water, runtime 4.5 min) to afford the title compound. (11 mg, 27% yield) as a light brown foam. LCMS: m/z 483.5 [M+H]⁺, ESI pos.

### Example 16

### 4-[(3R)-3-[[6-(4-Hydroxy-2,3-dihydrobenzofuran-5-yl)-5-methyl-1,2,4-triazin-3-yl]amino]-1-piperidyl]-1-morpholino-butan-1-one

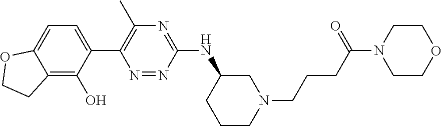 [SMILES: Cc1nc(N[C@@H]2CCCN(CCCC(=O)N3CCOCC3)C2)nnc1-c1ccc2c(c1O)CCO2]

To a solution of 4-[(3R)-3-[[6-(4-hydroxycoumaran-5-yl)-5-methyl-1,2,4-triazin-3-yl]amino]piperidino]butyric acid (Example 9, step E) (40 mg, 0.097 mmol, 1.0 eq) and N,N-dimethylformamide, extra dry (2.5 mL) was added at ambient temperature N,N-diisopropylethylamine (62.5 mg, 0.082 mL, 0.484 umol, 5.0 eq) followed by HATU (CAS #148893-10-1, 55.2 mg, 0.145 mmol, 1.5 eq). The yellow solution was stirred for 4 min, then morpholine (CAS #110-91-8, 15.2 mg, 0.015 mL, 0.174 mmol, 1.8 eq) was added. After 24 h N,N-diisopropylethylamine (62.5 mg, 0.082 mL, 0.484 mmol, 5.0 eq) and HATU (55.2 mg, 0.145. mmol, 1.5 eq) were added again. After 4 min of stirring morpholine (15.2 mg, 0.015 mL, 0.174 mmol, 1.8 eq) was added again. The reaction mixture was stirred at 23° C. for 45 h in total. The reaction mixture was quenched with 2 mL of water. The reaction mixture was extracted with water (~10 mL) and ethylacetate (~15 mL), then the aqueous layer were backextracted with ethylacetate (~15 mL). The organic layers were washed with brine (~10 mL), dried over sodium sulfate, filtered and concentrated in vacuo. The product was then purified by preparative HPLC (Column: Gemini NX, 12 nm, 5 μm, 100×30 mm; Condition: ACN/water+0.1% TEA; Gradient: ACN in water, runtime 4.5 min) to afford the title compound. (4 mg, 8% yield) as a light yellow freeze-dried solid. LCMS: m/z 483.5 [M+H]⁺, ESI pos.

### Example 17

### 5-[5-Methyl-3-[[(3R)-1-(2-pyrazol-1-ylethyl)-3-piperidyl]amino]-1,2,4-triazin-6-yl]-2,3-dihydrobenzofuran-4-ol

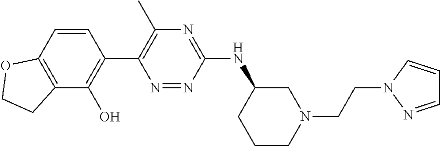 [SMILES: Cc1nc(N[C@@H]2CCCN(CCn3cccn3)C2)nnc1-c1ccc2c(c1O)CCO2]

### Step A: 6-Chloro-5-methyl-N-[(3R)-1-(2-pyrazol-1-ylethyl)-3-piperidyl]-1,2,4-triazin-3-amine

To a mixture of (6-chloro-5-methyl-1,2,4-triazin-3-yl)-[(3R)-3-piperidyl]amine (Example 1, step E) (250 mg, 1.1 mmol, 1.0 eq) in tetrahydrofuran, extra dry (4 mL) and N,N-dimethylformamide, extra dry (4 mL) was added N,N-diisopropylethylamine (568 mg, 0.7467 mL, 4.4 mmol, 4.0 eq) followed by addition of 1-(2-bromoethyl)pyrazole; hydrobromide (CAS #1955531-53-9, 443.7 mg, 1.65 mmol, 1.5 eq). The reaction mixture was stirred at 60° C. for 20 h. The reaction was quenched with water (20 mL) and extracted with ethyl acetate (2×60 mL). The organic layers were washed with brine (60 mL), dried over sodium sulfate, filtered off and concentrated in vacuo. The residue was purified by flash chromatography on silica gel (0% to 100% ethyl acetate in heptane) to afford the title compound (231 mg, 62% yield) as a light brown oil. LCMS: m/z 322.3 ([{35Cl}M+H]⁺), 324.2 ([{37Cl}M+H]⁺), ESI pos.

### Step B: 5-[5-Methyl-3-[[(3R)-1-(2-pyrazol-1-ylethyl)-3-piperidyl]amino]-1,2,4-triazin-6-yl]-2,3-dihydrobenzofuran-4-ol

A mixture of (6-chloro-5-methyl-1,2,4-triazin-3-yl)-[(3R)-1-(2-pyrazol-1-ylethyl)-3-piperidyl]-amine (Example 17, step A) (113 mg, 0.351 mmol, 1.0 eq), 5-(4,4,5,5-tetramethyl-1,3,2-dioxaborolan-2-yl)coumaran-4-ol (Example 1, step C) (128.9 mg, 0.492 mmol, 1.4 eq) and cesium carbonate (343 mg, 1.05 mmol, 3.0 eq) in 1,4-dioxane (4 mL) and water (1 mL) was set under argon. Finally XPhos Pd g3 gt (44.6 mg, 0.053 mmol, 0.15 eq) was added. The reaction mixture was stirred at 90° C. in a sealed tube for 3 hours. The reaction mixture was cooled to room temperature and quenched with water (20 mL) and aq. sat. NH₄Cl (20 mL), then extracted with ethyl acetate (2×40 mL). The organic layers were washed with brine (40 mL), dried over sodium sulfate, filtered off and concentrated in vacuo. The crude product was purified by flash chromatography on silica gel (gradient 0% to 80% (dichloromethane:methanol:NH₄OH 110:10:1) in dichloromethane) to afford the title compound (89 mg, 57% yield) as a yellow foam. LCMS: m/z 422.4 [M+H]⁺, ESI pos.

### Example 18 and 19

### 5-[5-Methyl-3-[[(3R)-1-[3-(1H-tetrazol-5-yl)propyl]-3-piperidyl]amino]-1,2,4-triazin-6-yl]-2,3-dihydrobenzofuran-4-ol; formic acid and 4-[(3R)-3-[[6-(4-Hydroxy-2,3-dihydrobenzofuran-5-yl)-5-methyl-1,2,4-triazin-3-yl]amino]-1-piperidyl]butanenitrile

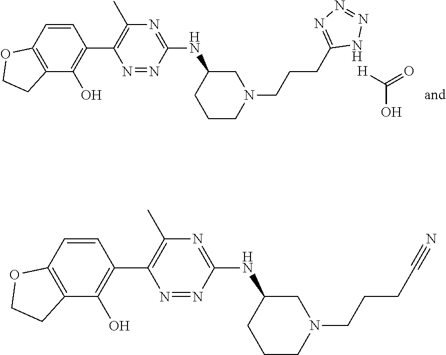

### Step A: 4-[(3R)-3-[(6-Hhloro-5-methyl-1,2,4-triazin-3-yl)amino]-1-piperidyl]butanenitrile

To a mixture of (6-chloro-5-methyl-1,2,4-triazin-3-yl)-[(3R)-3-piperidyl]amine (Example 1, step E) (400 mg, 1.76 mmol, 1.0 eq) in tetrahydrofuran, extra dry (6 mL) and N,N-dimethylformamide, extra dry (6 mL) was added N,N-diisopropylethylamine (567 mg, 0.747 mL, 4.4 mmol, 2.5 eq) followed by dropwise addition of 4-bromobutyronitrile (CAS #5332-06-9, 390 mg, 0.26 mL, 2.64 mmol, 1.5 eq). The reaction mixture was stirred at 60° C. for 16 hours. The reaction was quenched with water (20 mL) and extracted with ethyl acetate (2×60 mL). The organic layers were washed with brine (60 mL), dried over sodium sulfate, filtered off and concentrated in vacuo. The residue was purified by flash chromatography on silica gel (0% to 100% ethyl acetate in heptane) to afford the title compound (391 mg, 72% yield) as a light brown oil. LCMS: m/z 295.2 ([{35Cl}M+H]⁺), 297.2 ([{37Cl}M+H]⁺), ESI pos.

### Step B: 4-[(3R)-3-[[6-(4-Hydroxy-2,3-dihydrobenzofuran-5-yl)-5-methyl-1,2,4-triazin-3-yl]amino]-1-piperidyl]butanenitrile

A mixture of 4-[(3R)-3-[(6-chloro-5-methyl-1,2,4-triazin-3-yl)amino]piperidino]butyronitrile (Example 18, step A) (150 mg, 0.51 mmol, 1.0 eq), 5-(4,4,5,5-tetramethyl-1,3,2-dioxaborolan-2-yl)coumaran-4-ol (Example 1, step C) (187 mg, 0.712 mmol, 1.4 eq) and cesium carbonate (498 mg, 1.53 mmol, 3.0 eq) in 1,4-dioxane (4 mL) and water (1 mL) was set under argon, and XPhos Pd g3 gt (65 mg, 0.076 mmol, 0.15 eq) was added finally. The reaction mixture was stirred at 90° C. in a sealed tube for 3 hours. The reaction mixture was cooled to room temperature and quenched with water (20 mL) and aq. sat. NH₄Cl (20 mL), then extracted with ethyl acetate (2×40 mL). The organic layers were washed with brine (40 mL), dried over sodium sulfate, filtered off and concentrated in vacuo. The crude product was purified by flash chromatography on silica gel (gradient 0% to 80% (dichloromethane:methanol:NH₄OH 110:10:1) in dichloromethane) followed by a second purification by flash chromatography (SiO2-Amine; 0% to 100% ethyl acetate in heptane) to afford the title compound (145 mg, 69% yield) as a yellow foam. LCMS: m/z 395.2 [M+H]⁺, ESI pos.

### Step C: 5-[5-Methyl-3-[[(3R)-1-[3-(1H-tetrazol-5-yl)propyl]-3-piperidyl]amino]-1,2,4-triazin-6-yl]-2,3-dihydrobenzofuran-4-ol; formic acid

To a solution of 4-[(3R)-3-[[6-(4-hydroxycoumaran-5-yl)-5-methyl-1,2,4-triazin-3-yl]amino]piperidino]butyronitrile (Example 18, step B) (135 mg, 0.343 mmol, 1.0 eq) in N,N-dimethylformamide, extra dry (1.45 mL) was added sodium azide (CAS #26628-22-8, 100 mg, 1.54 mmol, 4.5 eq) followed by the addition of L-proline (CAS #147-85-3, 11.8 mg, 0.103 mmol, 0.3 eq). The reaction mixture was stirred in a sealed tube at 115° C. for 16 hours. After 16 h, more sodium azide (100 mg, 1.54 mmol, 4.5 eq) and L-proline (11.8 mg, 0.103 mmol, 0.3 eq) were added and stirring continued at 120° C. for another 60 hours. The reaction mixture was quenched with water (5 mL) and extracted with ethylacetate (2×30 mL). The aqueous phase was evaporated, the solid residues were triturated in DCM/MeOH 9:1, some sodium sulfate was added, then filtered off. The product was then purified by preparative HPLC (Column: YMC-Triart C18, 12 nm, 5 μm, 100×30 mm; Condition: ACN/water+0.1% HCOOH; Gradient 5-50% ACN, runtime 4.5 min) to afford the title compound (51 mg, 29% yield) as a light brown freeze-dried solid. LCMS: m/z 438.4 [M+H]⁺, ESI pos.

### Example 20

### 5-[5-Methyl-3-[[(3R)-1-(3-oxazol-2-ylpropyl)-3-piperidyl]amino]-1,2,4-triazin-6-yl]-2,3-dihydrobenzofuran-4-ol

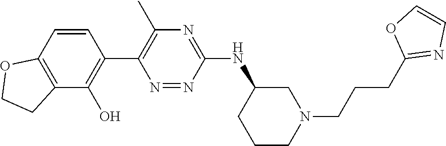 [SMILES: Cc1nc(N[C@@H]2CCCN(CCCc3ncco3)C2)nnc1-c1ccc2c(c1O)CCO2]

### Step A: 6-Chloro-5-methyl-N-[(3R)-1-(3-oxazol-2-ylpropyl)-3-piperidyl]-1,2,4-triazin-3-amine

To a solution of (6-chloro-5-methyl-1,2,4-triazin-3-yl)-[(3R)-3-piperidyl]amine 1:2 hydrogen chloride (Example 1, step B) (67 mg, 0.178 mmol, 1.0 eq) in dichloromethane, extra dry (2 mL) was added at 0° C. 3-oxazol-2-ylpropionaldehyde (CAS #1214937-88-8, 31.2 mg, 0.25 mmol, 1.4 eq) followed by sodium acetate (36.6 mg, 0.446 mmol, 2.0 eq) and sodium triacetoxyborohydride (CAS #56553-60-7, 68 mg, 0.321 mmol, 1.8 eq). The reaction mixture was stirred at 0° C. for 5 min and at room temperature for 3 hours. To the reaction mixture, saturated NaHCO₃ solution (20 mL) was added and extracted with dichloromethane (3×30 mL). The organic phase was separated and washed with water (20 mL) and brine (20 mL). The combined organic layers were dried over sodium sulfate, filtered off and concentrated in vacuo. The crude product was purified by flash chromatography on silica gel (gradient 0% to 50% (dichloromethane:methanol:NH₄OH 110:10:1) in dichloromethane) to afford the title compound (43 mg, 72% yield) as a light brown oil. LCMS: m/z 337.2 ([{35Cl}M+H]⁺), 339.2 ([{37Cl}M+H]⁺), ESI pos.

### Step B: 5-[5-Methyl-3-[[(3R)-1-(3-oxazol-2-ylpropyl)-3-piperidyl]amino]-1,2,4-triazin-6-yl]-2,3-dihydrobenzofuran-4-ol

A mixture of (6-chloro-5-methyl-1,2,4-triazin-3-yl)-[(3R)-1-(3-oxazol-2-ylpropyl)-3-piperidyl]amine (Example 20, step A) (43 mg, 0.128 mmol, 1.0 eq), 5-(4,4,5,5-tetramethyl-1,3,2-dioxaborolan-2-yl)coumaran-4-ol (Example 1, step C) (55.8 mg, 0.192 mmol, 1.5 eq) and cesium carbonate (125 mg, 0.383 mmol, 3.0 eq) in 1,4-dioxane (1.6 mL) and water (0.4 mL) was set under argon, and XPhos Pd G3 gt (16 mg, 0.019 mmol, 0.15 eq) was added finally. The reaction mixture was stirred at 90° C. in a sealed tube for 2 hours. The reaction mixture was cooled to room temperature and quenched with water (10 mL) and aq. sat. NH₄Cl (10 mL), then extracted with ethyl acetate (2×20 mL). The organic layers were washed with brine (20 mL), dried over sodium sulfate, filtered off and concentrated in vacuo. The crude product was purified by flash chromatography on silica gel (gradient 0% to 50% (dichloromethane:methanol:NH₄OH 110:10:1) in dichloromethane) to afford the title compound (19 mg, 32% yield) as a light yellow foam. LCMS: m/z 437.3 [M+H]⁺, ESI pos.

### Example 21

### 5-[5-Methyl-3-[[(3R)-1-[2-(1H-tetrazol-5-yl)ethyl]-3-piperidyl]amino]-1,2,4-triazin-6-yl]-2,3-dihydrobenzofuran-4-ol

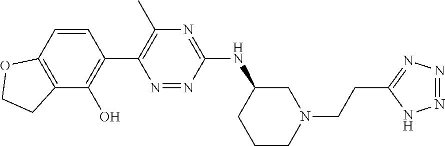 [SMILES: Cc1nc(N[C@@H]2CCCN(CCc3nnn[nH]3)C2)nnc1-c1ccc2c(c1O)CCO2]

### Step A: 3-[(3R)-3-[(6-Chloro-5-methyl-1.2,4-triazin-3-yl]aminol-1-piperidyl]propanenitrile

To a mixture of (6-chloro-5-methyl-1,2,4-triazin-3-yl)-[(3R)-3-piperidyl]amine (Example 1, step E) (500 mg, 2.2 mmol, 1.0 eq) in tetrahydrofuran, extra dry (8 mL) and N,N-dimethylformamide, extra dry (8 mL) was added N,N-diisopropylethylamine (710 mg, 0.934 mL, 5.5 mmol, 2.5 eq) followed by dropwise addition of 3-bromopropionitrile (CAS #2417-90-5, 465 mg, 0.287 mL, 3.3 mmol, 1.5 eq). The reaction mixture was stirred at 60° C. for 48 hours. The reaction was quenched with water (20 mL) and extracted with ethyl acetate (2×60 mL). The organic layers were washed with brine (60 mL), dried over Na₂SO₄, filtered off and concentrated in vacuo. The residue was purified by flash chromatography on silica gel (0% to 100% ethyl acetate in heptane) to afford the title compound (425 mg, 65% yield) as a light brown oil. LCMS: m/z 281.2 ([{35Cl}M+H]⁺), 283.2 ([{37Cl}M+H]⁺), ESI pos.

### Step B: 3-[(3R)-3-[[6-(4-Hydroxy-2,3-dihydrobenzofuran-5-yl)-5-methyl-1,2,4-triazin-3-yl]amino]-1-piperidyl]propanenitrile

A mixture of 3-[(3R)-3-[(6-chloro-5-methyl-1,2,4-triazin-3-yl)amino]piperidino]propionitrile (Example 22, step A) (200 mg, 0.712 mmol, 1.0 eq) was dissolved in 1,4-dioxane, extra dry (4 mL) and water (1 mL). Then 5-(4,4,5,5-tetramethyl-1,3,2-dioxaborolan-2-yl)coumaran-4-ol (Example 1, step C) (280 mg, 1.07 mmol, 1.5 eq), cesium carbonate (696 mg, 2.2 mmol, 3.0 eq) and XPhos Pd g3 (90.5 mg, 0.10 mmol, 0.15 eq) was added under an argon atmosphere. The reaction mixture was sealed and stirred at 100° C. for 1.5 hours. The reaction mixture was quenched with water (10 mL) and sat. aq. NH₄Cl (10 mL), then extracted with ethyl acetate (2×25 mL). The organic layers were washed with brine (15 mL), dried over Na₂SO₄, filtered off and concentrated in vacuo. The crude product was purified by flash chromatography on silica gel (gradient 0% to 80% (dichloromethane:methanol:NH₄OH 110:10:1) in dichloromethane) to afford the title compound (240 mg, 80% yield) as a light brown oil. LCMS: m/z 381.3 [M+H]⁺, ESI pos.

### Step C: 5-[5-Methyl-3-[[(3R)-1-[2-(1H-tetrazol-5-yl)ethyl]-3-piperidyl]amino]-1,2,4-triazin-6-yl]-2,3-dihydrobenzofuran-4-ol

To a solution of 3-[(3R)-3-[[6-(4-hydroxycoumaran-5-yl)-5-methyl-1,2,4-triazin-3-yl]amino]piperidino]propionitrile (Example 22, step B) (100 mg, 0.237 mmol, 1.0 eq) in N,N-dimethylformamide, extra dry (1 mL) was added L-proline (CAS #147-85-3, 8.2 mg, 0.071 μmol, 0.30 eq) followed by sodium azide (CAS #26628-22-8, 69 mg, 1.06 mmol, 4.5 eq). The reaction mixture was stirred at 115° C. for 16 hours. After 16 h, L-proline (8.2 mg, 0.071 mmol, 0.3 eq) and sodium azide (69 mg, 1.06 mmol, 4.5 eq) were added again. The reaction mixture was stirred at 115° C. for 48 hours. The reaction mixture was quenched with water (~5 mL) and then extracted with ethyl acetate (~25 mL). The product was in the aqueous phase. The aqueous layer was backextracted four times with ethyl acetate (~25 mL). The aqueous layer was concentrated in vacuo. The crude product was then purified by preparative HPLC (Column: YMC-Triart Cis, 12 nm, 5 μm, 100×30 mm; Condition: ACN/water+0.1% HCOOH; Gradient: 5-50% ACN, runtime 4.5 min) to afford the title (3 mg, 3% yield) as light brown freeze-dried solid. LCMS: m/z 242.3 [M+H]⁺, ESI pos.

### Example 22

### 5-[5-Methyl-3-[[(3R)-1-(3-methylsulfonylpropyl)-3-piperidyl]amino]-1,2,4-triazin-6-yl]-2,3-dihydrobenzofuran-4-ol

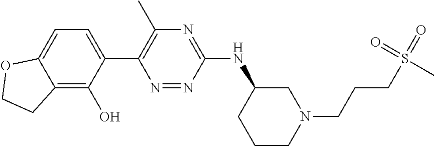 [SMILES: Cc1nc(N[C@@H]2CCCN(CCCS(C)(=O)=O)C2)nnc1-c1ccc2c(c1O)CCO2]

### Step A: 6-Chloro-5-methyl-N-[(3R)-1-(3-methylsulfonylpropyl)-3-piperidyl]-1,2,4-triazin-3-amine

To a mixture of (6-chloro-5-methyl-1,2,4-triazin-3-yl)-[(3R)-3-piperidyl]amine (Example 1, step E) (100 mg, 0.439 mmol, 1.0 eq) in tetrahydrofuran, extra dry (1.6 mL) and N,N-dimethylformamide, extra dry (1.6 mL) was added N,N-diisopropylethylamine (142.0 mg, 0.187 mL, 1.1 mmol, 2.5 eq) followed by dropwise addition of 1-bromo-3-mesyl-propane (CAS #859940-73-1, 132.5 mg, 0.659 mmol, 1.5 eq). The reaction mixture was stirred at 60° C. for 12 hours. The reaction was quenched with water (10 mL) and extracted with ethyl acetate (2×40 mL). The organic layers were washed with brine (20 mL), dried over sodium sulfate, filtered off and concentrated in vacuo. The residue was purified by flash chromatography (silica gel, 12 g; 0% to 10% MeOH in DCM) to afford the title compound (111 mg, 65% yield) as an orange oil. LCMS: m/z 348.2 [M+H]⁺, ESI pos.

### Step B: 5-[3-[[(3R)-1-(3-Mesylpropyl)-3-piperidyl]amino]-5-methyl-1,2,4-triazin-6-yl]coumaran-4-ol

A mixture of (6-chloro-5-methyl-1,2,4-triazin-3-yl)-[(3R)-1-(3-mesylpropyl)-3-piperidyl]amine (Example 23, step A) (108 mg, 0.310 mmol, 1.0 eq), 5-(4,4,5,5-tetramethyl-1,3,2-dioxaborolan-2-yl)coumaran-4-ol (Example 1, step C) (144.67 mg, 0.497 mmol, 1.6 eq), cesium carbonate (305 mg, 0.936 mmol, 3.0 eq) and XPhos Pd g3 (39.4 mg, 0.047 mmol, 0.15 eq) in 1,4-dioxane (1.9 mL) and water (0.46 mL) was flushed with argon and stirred at 90° C. for 3 h. The reaction mixture was cooled to room temperature and extracted with ethyl acetate and half-saturated NH₄Cl-solution. The aqueous layer was backextracted with ethyl acetate. The organic layers were washed with water and brine. The combined organic layers were dried over sodium sulfate, filtered and concentrated in vacuo. The crude product was purified by flash chromatography (Si-amine, 12 g, gradient 0% to 10% methanol in DCM) to afford the title compound (108 mg, 70% yield) as a yellow solid. LCMS: m/z 448.3 [M+H]⁺, ESI pos.

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula I 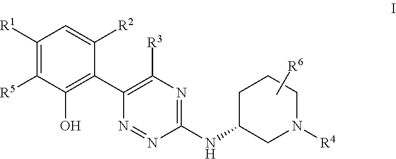
wherein
R¹ and R⁵, and the atoms to which they are bonded, form either
i. a 4-6 membered heterocycle ring comprising a single O heteroatom, or
ii. a 4-5 membered cycloalkyl ring;
R² and R³ are selected from H and alkyl, wherein only one of R² or R³ can be H or alkyl;
R⁴ is selected from
i. alkyl,
ii. hydroxyalkyl,
iii. alkoxyalkyl,
iv. cycloalkyl substituted with hydroxy or alkoxy,
v. cycloalkylalkyl substituted with hydroxyl, alkoxy, or —COOH,
vi. a 4-6 membered heterocycle comprising a single O heteroatom,
vii. (CH₂)ₙC(O)OH, (CH₂)ₙC(O)OCH₃, or (CH₂)ₙC(O)NHCH₃, wherein n is 2 or 3,
viii. heteroarylalkyl, wherein the heteroaryl in heteroarylalkyl is a tetrazole, oxadiazole or an oxazole heteroaryl,
ix. heteroarylalkyl, wherein the heteroaryl in heteroarylalkyl is a pyrazole;
x. —(CH₂)ₙS(O)₂CH₃ or (CH₂)ₙCN wherein n is 3,
xi. —(CH₂)ₙC(O)NR′R″, wherein n is 3 and R′ and R″ are both CH₃, R′ is CH₃ and R″ is a hydroxylalkyl, or R′ and R″ together with the N to which they are connected form either
a. a 5 member heterocycle ring, wherein the heterocycle ring is optionally substituted with OH, or
b. a 6 member heterocycle ring additionally comprising 1 O heteroatom;
R⁶ is H or —OH,
wherein R⁴ can only be alkyl when R⁶ is —OH;
and pharmaceutically acceptable salts.

2. A compound according to claim 1, wherein R¹ and R⁵, and the atoms to which they are bonded, form a 5-membered heterocycle ring comprising a single O heteroatom.

3. A compound according to claim 1 or claim 2, wherein R² is H and R³ is alkyl.

4. A compound according to any of claims 1 to 3, wherein R⁴ is selected from
i. alkyl,
ii. hydroxyalkyl,
iii. alkoxyalkyl,
iv. cycloalkylalkyl substituted with hydroxy,
v. a 4-membered heterocycle comprising a single O heteroatom,
vi. (CH₂)ₙC(O)OH, (CH₂)ₙC(O)OCH₃, or (CH₂)ₙC(O)NHCH₃, wherein n is 3,
vii. heteroarylalkyl, wherein the heteroaryl in heteroarylalkyl is an oxazole heteroaryl,
viii. heteroarylalkyl, wherein the heteroaryl in heteroarylalkyl is a pyrazole;
ix. —(CH₂)ₙS(O)₂CH₃ or (CH₂)ₙCN wherein n is 3,
x. —(CH₂)ₙC(O)NR′R″, wherein n is 3 and R′ and R″ are both CH₃, R′ is CH₃ and R″ is a hydroxylalkyl, or R′ and R″ together with the N to which they are connected form either
a. a 5 member heterocycle ring, wherein the heterocycle ring is optionally substituted with OH, or
b. a 6 member heterocycle ring additionally comprising 1 O heteroatom;
wherein R⁴ can only be alkyl when R⁶ is —OH.

5. A compound according to any of claims 1 to 3, wherein R⁴ is selected from
i. alkyl,
ii. hydroxyalkyl,
iii. alkoxyalkyl,
iv. cycloalkylalkyl substituted with hydroxy,
v. a 4-membered heterocycle comprising a single O heteroatom,
vi. (CH₂)ₙC(O)OH, (CH₂)ₙC(O)OCH₃, or (CH₂)ₙC(O)NHCH₃, wherein n is 3,
vii. heteroarylalkyl, wherein the heteroaryl in heteroarylalkyl is an oxazole heteroaryl;
wherein R⁴ can only be alkyl when R⁶ is —OH.

6. A compound according to any of claims 1 to 3, wherein R⁴ is selected from
i. cycloalkyl substituted with hydroxy or alkoxy,
ii. cycloalkylalkyl substituted with hydroxy or alkoxy, and
iii. a 4-6 membered heterocycle comprising a single O heteroatom.

7. A compound according to any of claims 1 to 3, wherein R⁴ is selected from
i. cycloalkyl substituted with hydroxy or alkoxy, and
ii. cycloalkylalkyl substituted with hydroxy or alkoxy.

8. A compound according to any of claims 1 to 7, wherein R⁶ is H.

9. A compound according to claim 1, wherein
R¹ and R⁵, and the atoms to which they are bonded, form a 5-membered heterocycle ring comprising a single O heteroatom;
R² is H and R³ is methyl;
R⁴ is selected from
i. alkyl,
ii. hydroxyalkyl,
iii. alkoxyalkyl,
iv. cycloalkylalkyl substituted with hydroxy,
v. a 4-membered heterocycle comprising a single O heteroatom,
vi. (CH₂)ₙC(O)OH, (CH₂)ₙC(O)OCH₃, or (CH₂)ₙC(O)NHCH₃, wherein n is 3,
vii. heteroarylalkyl, wherein the heteroaryl in heteroarylalkyl is an oxazole heteroaryl,
viii. heteroarylalkyl, wherein the heteroaryl in heteroarylalkyl is a pyrazole,
ix. —(CH₂)ₙS(O)₂CH₃ or (CH₂)ₙCN wherein n is 3,
x. —(CH₂)ₙC(O)NR′R″, wherein n is 3 and R′ and R″ are both CH₃, R′ is CH₃ and R″ is a hydroxylalkyl, or R′ and R″ together with the N to which they are connected form either
a. a 5 member heterocycle ring, wherein the heterocycle ring is optionally substituted with OH, or
b. a 6 member heterocycle ring additionally comprising 1 O heteroatom;
R⁶ is H or —OH,
wherein R⁴ can only be alkyl when R⁶ is —OH;
and pharmaceutically acceptable salts.

10. A compound according to claim 1, wherein
R¹ and R⁵, and the atoms to which they are bonded, form a 5-membered heterocycle ring comprising a single O heteroatom;
R² is H and R³ is methyl;
R⁴ is selected from
i. alkyl,
ii. hydroxyalkyl,
iii. alkoxyalkyl,
iv. cycloalkylalkyl substituted with hydroxy,
v. a 4-membered heterocycle comprising a single O heteroatom,
vi. (CH₂)ₙC(O)OH, (CH₂)ₙC(O)OCH₃, or (CH₂)ₙC(O)NHCH₃, wherein n is 3,
vii. heteroarylalkyl, wherein the heteroaryl in heteroarylalkyl is an oxazole heteroaryl;
R⁶ is H or —OH,
wherein R⁴ can only be alkyl when R⁶ is —OH;
and pharmaceutically acceptable salts.

11. A compound according to claim 1, wherein the compound is 5-[3-[[(3R)-1-(2-Hydroxyethyl)-3-piperidyl]amino]-5-methyl-1,2,4-triazin-6-yl]-2,3-dihydrobenzofuran-4-ol or pharmaceutically acceptable salts thereof.

12. A compound according to claim 1, wherein the compound is selected from
5-[5-Methyl-3-[[rac-(3R)-1-(2-hydroxypropyl)-3-piperidyl]amino]-1,2,4-triazin-6-yl]-2,3-dihydrobenzofuran-4-ol;
5-[3-[[(3R)-1-(3-Hydroxypropyl)-3-piperidyl]amino]-5-methyl-1,2,4-triazin-6-yl]-2,3-dihydrobenzofuran-4-ol;
5-[3-[[(3R)-1-[(1-Hydroxycyclopropyl)methyl]-3-piperidyl]amino]-5-methyl-1,2,4-triazin-6-yl]-2,3-dihydrobenzofuran-4-ol;
5-[3-[[(3R)-1-(2-Methoxyethyl)-3-piperidyl]amino]-5-methyl-1,2,4-triazin-6-yl]-2,3-dihydrobenzofuran-4-ol; formic acid salt;
5-[3-[[(3R)-1-(2-Methoxyethyl)-3-piperidyl]amino]-5-methyl-1,2,4-triazin-6-yl]-2,3-dihydrobenzofuran-4-ol;
(3S,5R)-1-Ethyl-5-[[6-(4-hydroxy-2,3-dihydrobenzofuran-5-yl)-5-methyl-1,2,4-triazin-3-yl]amino]piperidin-3-ol;
5-[5-Methyl-3-[[rac-(3R)-1-(2-methoxypropyl)-3-piperidyl]amino]-1,2,4-triazin-6-yl]-2,3-dihydrobenzofuran-4-ol;
5-[5-Methyl-3-[[(3R)-1-(oxetan-3-yl)-3-piperidyl]amino]-1,2,4-triazin-6-yl]-2,3-dihydrobenzofuran-4-ol;
4-[(3R)-3-[[6-(4-hydroxy-2,3-dihydrobenzofuran-5-yl)-5-methyl-1,2,4-triazin-3-yl]amino]-1-piperidyl]butanoic acid;
4-[(3R)-3-[[6-(4-hydroxy-2,3-dihydrobenzofuran-5-yl)-5-methyl-1,2,4-triazin-3-yl]amino]-1-piperidyl];
Methyl 4-[(3R)-3-[[6-(4-hydroxy-2,3-dihydrobenzofuran-5-yl)-5-methyl-1,2,4-triazin-3-yl]amino]-1-piperidyl]butanoate;
4-[(3R)-3-[[6-(4-hydroxy-2,3-dihydrobenzofuran-5-yl)-5-methyl-1,2,4-triazin-3-yl]amino]-1-piperidyl]-N-methyl-butanamide;
5-[5-methyl-3-[[(3R)-1-(2-oxazol-2-ylethyl)-3-piperidyl]amino]-1,2,4-triazin-6-yl]-2,3-dihydrobenzofuran-4-ol;
and pharmaceutically acceptable salts thereof.

13. A compound according to claim 1, wherein the compound is selected from
4-[(3R)-3-[[6-(4-hydroxy-2,3-dihydrobenzofuran-5-yl)-5-methyl-1,2,4-triazin-3-yl]amino]-1-piperidyl]-N,N-dimethyl-butanamide;
4-[(3R)-3-[[6-(4-hydroxy-2,3-dihydrobenzofuran-5-yl)-5-methyl-1,2,4-triazin-3-yl]amino]-1-piperidyl]-1-pyrrolidin-1-yl-butan-1-one;
4-[(3R)-3-[[6-(4-hydroxy-2,3-dihydrobenzofuran-5-yl)-5-methyl-1,2,4-triazin-3-yl]amino]-1-piperidyl]-N-(2-hydroxyethyl)-N-methyl-butanamide;
1-(3-hydroxypyrrolidin-1-yl)-4-[rac-(3R)-3-[[6-(4-hydroxy-2,3-dihydrobenzofuran-5-yl)-5-methyl-1,2,4-triazin-3-yl]amino]-1-piperidyl]butan-1-one; formic acid;
1-(3-hydroxypyrrolidin-1-yl)-4-[rac-(3R)-3-[[6-(4-hydroxy-2,3-dihydrobenzofuran-5-yl)-5-methyl-1,2,4-triazin-3-yl]amino]-1-piperidyl]butan-1-one;
4-[(3R)-3-[[6-(4-hydroxy-2,3-dihydrobenzofuran-5-yl)-5-methyl-1,2,4-triazin-3-yl]amino]-1-piperidyl]-1-morpholino-butan-1-one;
5-[5-methyl-3-[[(3R)-1-(2-pyrazol-1-ylethyl)-3-piperidyl]amino]-1,2,4-triazin-6-yl]-2,3-dihydrobenzofuran-4-ol;
5-[5-methyl-3-[[(3R)-1-[3-(1H-tetrazol-5-yl)propyl]-3-piperidyl]amino]-1,2,4-triazin-6-yl]-2,3-dihydrobenzofuran-4-ol; formic acid;
5-[5-methyl-3-[[(3R)-1-[3-(1H-tetrazol-5-yl)propyl]-3-piperidyl]amino]-1,2,4-triazin-6-yl]-2,3-dihydrobenzofuran-4-ol;
4-[(3R)-3-[[6-(4-hydroxy-2,3-dihydrobenzofuran-5-yl)-5-methyl-1,2,4-triazin-3-yl]amino]-1-piperidyl]butanenitrile;
5-[5-methyl-3-[[(3R)-1-(3-oxazol-2-ylpropyl)-3-piperidyl]amino]-1,2,4-triazin-6-yl]-2,3-dihydrobenzofuran-4-ol;
4-[(3R)-3-[[6-(4-hydroxy-2,3-dihydrobenzofuran-5-yl)-5-methyl-1,2,4-triazin-3-yl]amino]-1-piperidyl]-N,N-dimethyl-butanamide; formic acid” 4-[(3R)-3-[[6-(4-hydroxy-2,3-dihydrobenzofuran-5-yl)-5-methyl-1,2,4-triazin-3-yl]amino]-1-piperidyl]-N,N-dimethyl-butanamide; formic acid;
5-[5-methyl-3-[[(3R)-1-[2-(1H-tetrazol-5-yl)ethyl]-3-piperidyl]amino]-1,2,4-triazin-6-yl]-2,3-dihydrobenzofuran-4-ol;
5-[5-methyl-3-[[(3R)-1-(3-methylsulfonylpropyl)-3-piperidyl]amino]-1,2,4-triazin-6-yl]-2,3-dihydrobenzofuran-4-ol;
and pharmaceutically acceptable salts thereof.

14. A compound according to claim 1, wherein the compound is 4-[(3R)-3-[[6-(4-hydroxy-2,3-dihydrobenzofuran-5-yl)-5-methyl-1,2,4-triazin-3-yl]amino]-1-piperidyl]butanoic acid, or pharmaceutically acceptable salts thereof.

15. A compound according to claim 1, wherein the compound is selected from
5-[5-methyl-3-[[(3R)-1-[3-(1H-tetrazol-5-yl)propyl]-3-piperidyl]amino]-1,2,4-triazin-6-yl]-2,3-dihydrobenzofuran-4-ol;
4-[(3R)-3-[[6-(4-hydroxy-2,3-dihydrobenzofuran-5-yl)-5-methyl-1,2,4-triazin-3-yl]amino]-1-piperidyl]-N,N-dimethyl-butanamide;
4-[(3R)-3-[[6-(4-hydroxy-2,3-dihydrobenzofuran-5-yl)-5-methyl-1,2,4-triazin-3-yl]amino]-1-piperidyl]-1-pyrrolidin-1-yl-butan-1-one;
and pharmaceutically acceptable salts thereof.

16. A compound according to any one of claims 1 to 15 for use as a therapeutically active substance.

17. A compound according to any one of claims 1 to 15 for use in the treatment or prevention of a disease, disorder or condition, wherein the disease, disorder or condition is responsive to NLRP3 inhibition.

18. A pharmaceutical composition comprising a compound according to any one of claims 1 to 15 and a therapeutically inert carrier.

19. The use of a compound according to any one of claims 1 to 15 for the treatment or prophylaxis of a disease, disorder or condition, wherein the disease, disorder or condition is responsive to NLRP3 inhibition.

20. A compound according to any one of claims 1 to 15 for use in the treatment or prophylaxis of a cardiovascular disease, disorder or condition.

21. The use of a compound according to any one of claims 1 to 15 in the treatment or prophylaxis of a cardiovascular disease, disorder or condition.

22. The use of a compound according to any one of claims 1 to 15 for the preparation of a medicament for the treatment or prophylaxis of a cardiovascular disease, disorder or condition.

23. A method of inhibiting NLRP3, which method comprises administering an effective amount of a compound as claimed in any one of claims 1 to 15 to inhibit NLRP3.

24. A method for the treatment or prophylaxis of a cardiovascular disease, disorder or condition, which method comprises administering an effective amount of a compound according to any one of claims 1 to 15.

25. The invention as hereinbefore described.
